(19) United States
(12) Patent Application Publication
(10) Pub. No.: US 2024/0237941 A1
(43) Pub. Date: Jul. 18, 2024

(54) SYSTEM, METHOD AND COMPUTER PROGRAM FOR MONITORING HEALTH OF A PERSON

(71) Applicant: MERU HEALTH OY, Helsinki (FI)

(72) Inventors: Kristian RANTA, Helsinki (FI); Albert NAZANDER, Helsinki (FI)

(21) Appl. No.: 18/554,491

(22) PCT Filed: Apr. 11, 2022
(86) PCT No.: PCT/FI2022/050236

§ 371 Date: Oct. 9, 2023

(30) Foreign Application Priority Data
Apr. 12, 2021 (FI) 20215426

(51) Int. Cl. A61B 5/00; A61B 5/024; G16H 40/67

(52) U.S. Cl. CPC A61B 5/486; A61B 5/02405; G16H 40/67

(57) ABSTRACT

A system for monitoring health of a person during a health monitoring session is disclosed. A breathing pacer is configured to time breathing events to the person with a breathing rate sequence including breathing rates associated with time windows (w1, w2), each including a timepoint (t1, t2). A periodic biofeedback function is fitted into the instantaneous heart rate data to determine the parameters of the periodic biofeedback function at each of the timepoints (t1, t2), and the cumulative difference E at each of the timepoints (t1, t2) to derive an optimal breathing frequency for vagus nerve stimulation from the parameters and from the cumulative difference E. A related method and a computer program are also disclosed.

### FIELD OF THE INVENTION

The present invention relates to a system for monitoring health of a person and particularly to a system according to preamble of claim 1. The present invention relates also to a method for monitoring health of a person and particularly to a method according to preamble of claim 23. The present invention relates also to a computer program for monitoring health of a person and particularly to a computer program according to preamble of claim 37.

### BACKGROUND OF THE INVENTION

Vagus nerve is an important nerve in the human body and it plays a major role in the correct functioning of the parasympathetic nervous system. In the medical community, stimulation of the vagus nerve is believed to lead to various cardiovascular, cerebrovascular, metabolic and other physiological and mental health benefits.

In the prior art, vagus nerve has been stimulated in a non-invasive way for example by application of electricity to the frontal neck area of the human body. Devices to this purpose are commercially available, and they resemble an electrical razor apparatus in size and shape. Also invasive ways of vagus nerve stimulation exist. As an example, a device may be surgically implanted under the skin of a person's chest, and a wire may be threaded under the skin to connect the device to the vagus nerve. When activated, the device sends electrical signals along the vagus nerve to person's brainstem, activating the vagus nerve and the nervous system in general.

It is also known that breathing in a certain, in general, slow breathing rate may stimulate the vagus nerve favourably. As one way to stimulate the vagus nerve favourably, breathing with a certain slow breathing rate, during a so-called breathing exercise, offers many advantages. Breathing correctly by a person involves no invasive or non-invasive electrical stimulation of the nerve which can be difficult to arrange especially if the related arrangements require surgical procedures or dedicated electrical devises or both. However, monitoring the breathing with the prior art devices and systems is challenging as monitoring breathing requires its own set of dedicated devices. These devices may be based, for example, to the analysis of flow of breathing inhalations and exhalations and to the movement of breathing gasses in the respiratory tract, for example mouth or nose. Clearly, wearing or holding such metering devices in mouth or by the nose is relatively unpleasant and complex.

It is believed that an effective positive vagus nerve stimulus through breathing is achieved when breathing rate of a person follows the natural heart rate variation of the person. In the medical community, this kind of stimulation is sometimes called “cross-coherence” between breathing and heart rate variation. In general, when a person inhales, the instantaneous heart rate increases, and when a person exhales, the instantaneous heart rate decreases.

Normally at rest, an average heart rate is between 60 and 100 BPM (beats per minute). However, this is heavily dependent on the gender, age, fitness level and level of relaxation of the person. Heart rate variations and breathing are also personal traits and there is no one universal value for a correct breathing rate to achieve strong vagus nerve stimulus through cross-coherence. Instead, every person has his or her own best breathing rate that best matches the heart rate variability (“HRV”) and breathing. Usually, the breathing rate that may provide strongest vagus nerve stimulus is between 3 and 8 breaths in one minute. In general, a high variability in the heart rate may be an indication of a strong vagus nerve stimulation with positive health effects.

In the prior art, there are some notions of analysing the cross-coherence of breathing rate and heartbeat rate of a person and of giving information to the person to find the best breathing rate to reach a high heart rate variability that may indicate strong vagus nerve stimulation. This has involved the usage of Fourier transforms and complex signal processing techniques to indicate spectral characteristics of heart rate variation and breathing. To get reliable spectral data, a very long dataset comprising data of instantaneous heart rates and breathing spanning several minutes has been needed. This makes the practical implementation of the system very difficult as the person whose breathing and heartbeats are monitored needs to remain in complete rest, and at the same time, pay attention to the way he/she breathes and, possibly, alter the way he/she breathes for several minutes.

Preferably, vagus nerve stimulation through breathing is attempted frequently, somewhat like a repeated fitness routine or exercise, as the positive health effects may grow gradually stronger. With repeated breathing exercises, a person may achieve the health benefits faster during the exercise. Optimal breathing rate for vagus nerve stimulation may also decrease somewhat over time. Clearly, a comfortable and fast approach for determining the heart rate variation, and a related indication of the strength of the related vagus nerve stimulation through breathing is needed.

Thus, at least two prior art problems can be raised: Stimulating the vagus nerve requires complex devices and procedures and there is a clear need to overcome the above-mentioned problems in the prior art. If vagus nerve is to be stimulated with a correct breathing rate and through breathing in general, determining the breathing rate well suited for a person to increase heart rate variability that may stimulate the vagus nerve favourably with prior art methods and devices has been challenging.

### BRIEF DESCRIPTION OF THE INVENTION

An object of the present invention is to provide a system, a method and a computer program for monitoring health of a person during a health monitoring session so that the prior art disadvantages are solved or at least alleviated. The objects of the invention are achieved by a system according to the independent claim 1. The objects of the invention are further achieved by a method according to the independent claim 23. The objects of the invention are further achieved by a computer program according to the independent claim 37.

The preferred embodiments of the invention are disclosed in the dependent claims.

The present invention is based on an idea of providing a system for monitoring health of a person during a health monitoring session. The system comprises:
a heart rate sensor arranged to measure heart beats of the person for providing heart rate measurement data,
a breathing pacer configured to time breathing events to the person with a breathing rate sequence comprising different breathing rates, the health monitoring session comprising a time window sequence, the time window sequence comprising time windows, such that each of the different breathing rates of the breathing rate sequence timed by the breathing pacer is associated with associated time window of the time window sequence,

the system comprises
a data processing system configured, during a health monitoring session, to
   A) receive the heart rate measurement data from the heart rate sensor,
   B) arrange the breathing pacer to time the breathing events to the person in each of the different breathing rates,
   C) analyse the heart rate measurement data such that in the analysis, the data processing system is configured to:
      C1) calculate instantaneous heart rate data comprising heart rate of each measured heartbeat, calculating performed over each of the time windows in the health monitoring session, each of the time windows comprising a timepoint, the instantaneous heart rate data being calculated based on the heart rate measurement data received from the heart rate sensor,
      C2) provide a periodic biofeedback function comprising parameters and a cumulative difference E relative to the instantaneous heart rate data, the parameters of the periodic biofeedback function comprising an amplitude A,
      C3) fit the periodic biofeedback function into the instantaneous heart rate data within each of the time windows to determine,
      the parameters of the periodic biofeedback function at each of the timepoints, and
      the cumulative difference E at each of the timepoints;
and
   E) determine a resonance score based on the amplitude A of the periodic biofeedback function and from the cumulative difference E within each of the time windows.

Advantage of the system is that a breathing frequency of the person that strongly stimulates the vagus nerve is determined quickly when compared to prior art systems, without complex arrangements in the respiratory tract. As an example, a timed and controlled breathing frequency sequence which is decreased down from a high breathing rate (for example 8 breaths/minute) a low one (for example 3 breaths/minute) at for example 0.5 breaths/minute intervals gives a broad and comprehensive dataset. It is straightforward to determine an optimal vagus nerve stimulation from this dataset.

In some embodiments, the data processing system is further configured, during a health monitoring session, to
D) store the parameters of the periodic biofeedback function, and the cumulative difference E at each of the timepoints.

In some embodiments, the data processing system is further configured, during a health monitoring session, to
F) compare the resonance scores of the time windows to each other and determine the breathing rate having the best resonance score.

In some embodiments, successive time windows in the time window sequence have different breathing rates timed by the breathing pacer.

Alternatively, successive time windows in the time window sequence have decreasing breathing rates timed by the breathing pacer.

Further alternatively, successive time windows in the time window sequence have increasing breathing rates timed by the breathing pacer.

In some embodiments, the data processing system is further configured, during a health monitoring session, to
B1) arrange the breathing pacer to operate at a first breathing rate in a first time window;
C1) analyse the heart rate measurement data within the first time window;
E1) determine the resonance score based on the amplitude A of the periodic biofeedback function and from the cumulative difference E within the first window;
C2) decrease the breathing rate of the breathing pacer and arrange the breathing pacer to operate at a decreased breathing rate in a subsequent time window;
C2) analyse the heart rate measurement data within the subsequent time window;
E2) determine the resonance score based on the amplitude A of the periodic biofeedback function and from the cumulative difference E within the subsequent window; and
F) compare the resonance scores of the first time window and the subsequent time window to each other and determine the breathing rate having the best resonance score.

Alternatively, the data processing system is further configured, during a health monitoring session, to
B1) arrange the breathing pacer to operate at a first breathing rate in a first time window;
C1) analyse the heart rate measurement data within the first time window;
E1) determine the resonance score based on the amplitude A of the periodic biofeedback function and from the cumulative difference E within the first window;
B2) decrease the breathing rate of the breathing pacer and arrange the breathing pacer to operate at a decreased breathing rate in a subsequent time window;
C2) analyse the heart rate measurement data within the subsequent time window;
E2) determine the resonance score based on the amplitude A of the periodic biofeedback function and from the cumulative difference E within the subsequent window;
repeat B2, C2 and E2 one or more time for two or more subsequent time windows in the time window sequence;
F) compare the resonance scores of the first time window and the subsequent time windows to each other and determine the breathing rate having the best resonance score.

In some embodiments, the data processing system is further configured, during a health monitoring session, to
B1) arrange the breathing pacer to operate at a first breathing rate in a first time window;
C1) analyse the heart rate measurement data within the first time window;
E1) determine the resonance score based on the amplitude A of the periodic biofeedback function and from the cumulative difference E within the first window;
C2) increase the breathing rate of the breathing pacer and arrange the breathing pacer to operate at a increased breathing rate in a subsequent time window;
C2) analyse the heart rate measurement data within the subsequent time window;
E2) determine the resonance score based on the amplitude A of the periodic biofeedback function and from the cumulative difference E within the subsequent window; and
F) compare the resonance scores of the first time window and the subsequent time window to each other and determine the breathing rate having the best resonance score.

In some alternative embodiments, the data processing system is further configured, during a health monitoring session, to
B1) arrange the breathing pacer to operate at a first breathing rate in a first time window;
C1) analyse the heart rate measurement data within the first time window;
E1) determine the resonance score based on the amplitude A of the periodic biofeedback function and from the cumulative difference E within the first window;
B2) increase the breathing rat of the breathing pacer and arrange the breathing pacer to operate at a increased breathing rate in a subsequent time window;
C2) analyse the heart rate measurement data within the subsequent time window;
E2) determine the resonance score based on the amplitude A of the periodic biofeedback function and from the cumulative difference E within the subsequent window;
repeat B2, C2 and E2 one or more time for two or more subsequent time windows in the time window sequence.
F) compare the resonance scores of the first time window and the subsequent time windows to each other and determine the breathing rate having the best resonance score.

In an embodiment, in the fitting, the data processing system is configured to: determine the cumulative difference E between the instantaneous heart rate data and the periodic biofeedback function within each of the time windows, the time windows comprising the timepoints, and perform the fitting based on a calculated minimum of the cumulative difference E within each of the time windows to determine the parameters of the periodic biofeedback function at each of the timepoints, the time windows comprising the timepoints.

Fitting a periodic function (here, the periodic biofeedback function) to a measurement data (here, to instantaneous heart rate data) based on a minimum of a cumulative difference is an effective way to achieve the fitting.

In an embodiment, the data processing system is configured perform the fitting within each of the time windows with a least squares method; or a modified least squares method; or a random search; or an exhaustive search; or any combination thereof. These fitting methods are effective methods for the fitting and they may be used in combination to avoid the convergence of the fitting method to a local minimum of the cumulative difference instead of a global minimum.

In an embodiment, in fitting, in determining the parameter indicating the amplitude A of the periodic biofeedback function at each of the timepoints, the data processing system is arranged to determine a minimum instantaneous heart rate and a maximum instantaneous heart rate of instantaneous heart rate data within each of the time windows, subtract the minimum instantaneous heart rate from the maximum instantaneous heart rate divide the value of subtraction by 2, and determine the parameter indicating the amplitude A of the periodic biofeedback function at each of the timepoints based on the value of the division. For a periodic, alternating function such as the periodic biofeedback function this is an effective way to determine an amplitude parameter of the function, or an initial guess for the amplitude parameter of the function.

In an embodiment, the parameters of the periodic biofeedback function comprise an angular frequency P, and in fitting, in determining the parameter indicating the angular frequency P of the periodic biofeedback function at each of the timepoints, the data processing system is arranged to determine a cycle time TD of the instantaneous heart rate data within each of the time windows, and determine the parameter indicating the angular frequency P of the periodic biofeedback function at each of the timepoints based on the cycle time TD. For a periodic, alternating function this is an effective way to determine an angular frequency parameter of the function, or an initial guess for the angular frequency parameter of the function.

In an embodiment, the parameters of the periodic biofeedback function comprise a mean M (or mean value M) of the periodic biofeedback function and in fitting, in determining the mean M of the periodic biofeedback function at each of the timepoints, the data processing system is arranged to determine a mean value of the instantaneous heart rate data within each of the time windows, and determine the mean M of the periodic biofeedback function at each of the timepoints based on the mean value. For a periodic, alternating function such as the periodic biofeedback function this is an effective way to determine the parameter indicating the mean of the function, or an initial guess for the parameter indicating the mean of the function.

In an embodiment, the periodic biofeedback function f(t) at each of the timepoints comprises a sinusoidal function sin(l such that f(t) is defined as f(t)=A sin(Pt−T)+M, in which A is the amplitude A of the instantaneous heart rate data within each of the time windows, P is the angular frequency P of the instantaneous heart rate data within each of the time windows, T is a time displacement, and M is a mean value M of the instantaneous heart rate data within each of the time windows. A sinusoidal function is able to represent the variation of the instantaneous heart rate well and is readily computed with digital means.

In an embodiment, the periodic biofeedback function f(t) at each of the timepoints comprises a skewed sinusoidal function sk sin( ) such that f(t) is defined as f(t)=A sk sin(Pt−T)+M=A sin[(Pt−T)+k*sin(Pt−T)], in which A is the amplitude A of the instantaneous heart rate data within each of the time windows, P is the angular frequency P of the instantaneous heart rate data within each of the time windows, T is a time displacement, k is a skew factor, * is a multiplication operator, and M is a mean value M of the instantaneous heart rate data within each of the time windows. A skewed sinusoidal function is also able to take into account the asymmetry of the inhale-exhale cycle of breathing and related heart rate variation as inhalation is often shorter than exhalation.

In an embodiment, the data processing system is configured to analyse the heart rate measurement data for each heartbeat measured with the heart rate sensor. This provides the most accurate set of data into which the periodic biofeedback function may be fitted.

In an embodiment, the breathing pacer is configured to provide feedback to the person within at least one of the time windows based on: the parameters of the periodic biofeedback function, or the cumulative difference E, or any combination thereof. A good fitting with a high amplitude parameter A, and small cumulative difference E is indicative of strong vagus nerve stimulation.

In an embodiment, the data processing system is configured to determine a resonance score from the amplitude A of the periodic biofeedback function and from the cumulative difference E at least within one of the time windows, and the breathing pacer is configured to provide feedback to the person within at least one of the time windows based on the resonance score. In another embodiment, the data processing system is configured to determine a resonance score from the amplitude A of the periodic biofeedback function and from the cumulative difference E within each of the time windows, and the breathing pacer is configured to provide feedback to the person by indicating to the person the breathing rate having the best resonance score. These are advantageous ways in delivering information on breathing rate which provides the strongest vagus nerve stimulation, the breathing rate determined, for example, in the ramp-down or ramp-up of the instructed breathing frequencies.

In an embodiment, the breathing pacer is configured to provide feedback to the person within at least one of the time windows visually, or audially, or haptically, or any combination thereof. These are advantageous ways in delivering information on how well the breathing stimulates the vagus nerve.

In an embodiment, the breathing pacer comprises third software means executable on a mobile computing device, and the third software means are functionally connectable with the data processing system, and the third software means comprises computer-executable instructions for providing feedback based on the parameters of the periodic biofeedback function, or the cumulative difference or any combination thereof. A mobile computing device like a smartphone or a tablet computer is an advantageous unit for implementing the functionality of the breathing pacer.

In an embodiment, the breathing pacer is arranged to time the breathing events to the person by indicating to the person a start of an inhalation of each breathing cycle; or an end of an inhalation of each breathing cycle; or a start of an exhalation of each breathing cycle; or an end of an exhalation of each breathing cycle; or any combination thereof. Many points in the breathing cycle are viable when instructing the person to breathe in an externally timed fashion.

In an embodiment, the data processing system comprises first software means executable on a mobile computing device, the first software means being functionally connectable with the heart rate sensor, and the first software means comprises computer-executable instructions to receive the heart rate measurement data, arrange the breathing pacer to time the breathing events, analyse the heart rate measurement data, and store the parameters and the cumulative difference. A mobile computing device like a smartphone or a tablet computer is an advantageous unit for implementing the functionality of data processing system.

In an embodiment, the data processing system comprises second software means executable on a network data server, the first software means and the second software means are configured to exchange data over a network connection, and the second software means comprise computer-executable instructions for performing at least one of the steps of receive the heart rate measurement data, arrange the breathing pacer to time the breathing events, analyse the heart rate measurement data, and store the parameters and the cumulative difference. A data server which is, for example, implemented in a “cloud” or a server cluster in a computer also an advantageous unit for analysing the heart rate measurements with a suitably fast network connection, for example a WLAN or a 3GPP connection.

In an embodiment, the second software means comprises computer-executable instructions for displaying the parameters of the periodic biofeedback function and the cumulative difference. A data server can provide output functionality of the analysis results, for example through a personal webpage or an applet of a mobile computing unit like smartphone.

In an embodiment, the breathing pacer is configured to time the breathing events to the person with the breathing rate sequence comprising breathing rates such that a successive breathing rate of the breathing rate sequence is lower than a previous breathing rate of the breathing rate sequence.

In an embodiment, the breathing pacer is configured to time the breathing events to the person with the breathing rate sequence comprising breathing rates such that a successive breathing rate of the breathing rate sequence is higher than a previous breathing rate of the breathing rate sequence.

In an embodiment, the breathing pacer is configured to time the breathing events to the person with the breathing rate sequence comprising breathing rates such that the breathing rate sequence is a predetermined breathing rate sequence.

Said three breathing rate sequences above are advantageous for iterating the best resonance score indicating the strongest vagus nerve stimulation for the person.

The present invention is also based on an idea of providing a method for monitoring health of a person during a health monitoring session. The method comprises:
measuring heart beats of the person for providing heart rate measurement data with a heart rate sensor,
receiving the heart rate measurement data from the heart rate sensor into a data processing system,
timing breathing events to the person with a breathing pacer and with a breathing rate sequence comprising different breathing rates, the health monitoring session comprising a time window sequence of time windows, such that each of the different breathing rates of the breathing rate sequence timed by the breathing pacer is associated with associated time window of the time window sequence,
analysing the heart rate measurement data in the data processing system by
   calculating instantaneous heart rate data comprising heart rate of each measured heartbeat, calculating performed over each of the time windows in the health monitoring session, each of the time windows comprising a timepoint, the instantaneous heart rate data being calculated based on the heart rate measurement data received from the heart rate sensor,
   providing a periodic biofeedback function comprising parameters, the parameters comprising an amplitude A, the periodic biofeedback function comprising a cumulative difference E relative to the instantaneous heart rate data,
   fitting the periodic biofeedback function into the instantaneous heart rate data within each of the time windows to determine the parameters of the periodic biofeedback function at each of the timepoints, and the cumulative difference E at each of the timepoints, and
determining a resonance score from the amplitude A of the periodic biofeedback function and from the cumulative difference E within each of the time windows.

In some embodiments, the method comprises storing the parameters of the periodic biofeedback function, and the cumulative difference E at each of the timepoints.

In some embodiments, the method comprises comparing the resonance scores of the time windows to each other and determine the breathing having the best resonance score.

In some embodiments, the method comprises providing successive time windows in the time window sequence have different breathing rates timed by the breathing pacer.

In some alternative embodiments, the method comprises providing successive time windows in the time window sequence have decreasing breathing rates timed by the breathing pacer.

In some further alternative embodiments, the method comprises providing successive time windows in the time window sequence have increasing breathing rates timed by the breathing pacer.

Advantage of the method is that a breathing frequency of the person that strongly stimulates the vagus nerve is determined quickly when compared to prior art systems, without complex arrangements in the respiratory tract. For example, a timed and controlled breathing frequency sequence which is ramped down from a high breathing rate (for example 8 breaths/minute) a low one (for example 3 breaths/minute) at for example 0.5 breaths/minute intervals gives a broad and comprehensive dataset. An optimal, personal breathing frequency that provides the strongest vagus nerve stimulation is straightforward to determine from this dataset.

In an embodiment, in the fitting, the method comprises determining the cumulative difference between the instantaneous heart rate data and the periodic biofeedback function within each of the time windows, the time windows comprising the timepoints, and performing the fitting based on a calculated minimum of the cumulative difference within each of the time windows to determine the parameters of the periodic biofeedback function at each of the timepoints, the time windows comprising the timepoints. Fitting a periodic function (here, the periodic biofeedback function) to a measurement data (here, to instantaneous heart rate data) based on a minimum of a cumulative difference is an effective way to achieve the fitting.

In an embodiment, the fitting is performed with a least squares method; or a modified least squares method; or a random search; or an exhaustive search; or any combination thereof. These fitting methods are effective methods for the fitting and they may be also used in combination to avoid the convergence of the fitting method to a local minimum of the cumulative difference instead of a global minimum.

In an embodiment, the periodic biofeedback function f(t) at each of the timepoints comprises a sinusoidal function sin( ) such that f(t) is defined as f(t)=A sin(Pt−T)+M, in which A is the amplitude A of the instantaneous heart rate data within each of the time windows, P is the angular frequency P of the instantaneous heart rate data within each of the time windows, T is a time displacement, and M is a mean value M of the instantaneous heart rate data within each of the time windows. A sinusoidal function is able to represent the variation of the instantaneous heart rate well and is readily computed with digital means.

In an embodiment, the periodic biofeedback function f(t) at each of the timepoints comprises a skewed sinusoidal function sk sin( ) such that f(t) is defined as f(t)=A sk sin(Pt−T)+M=A sin[(Pt−T)+k*sin(Pt−T)], in which A is the amplitude A of the instantaneous heart rate data within each of the time windows, P is the angular frequency P of the instantaneous heart rate data within each of the time windows, T is a time displacement, k is a skew factor, * is a multiplication operator, and M is a mean value M of the instantaneous heart rate data within each of the time windows. A skewed sinusoidal function is also able to take into account the asymmetry of the inhale-exhale cycle of breathing and related heart rate variation as inhalation is often shorter than exhalation.

In an embodiment, the method comprises providing feedback with the breathing pacer within at least one of the time windows, the feedback based on the parameters of the periodic biofeedback function, or the cumulative difference E, or any combination thereof. A good fitting indicated for example with a high amplitude and small cumulative difference is indicative of strong vagus nerve stimulation.

In an embodiment, the method comprises determining a resonance score from the amplitude of the periodic biofeedback function and from the cumulative difference within at least one of the time windows, and providing feedback to the person within at least one of the time windows based on the resonance score with the breathing pacer. In another embodiment, the method comprises determining a resonance score from the amplitude A of the periodic biofeedback function and from the cumulative difference E within each of the time windows, and providing feedback to the person by indicating to the person, with the breathing pacer, the breathing rate having the best resonance score. These are advantageous ways in delivering information on breathing rate which provides the strongest vagus nerve stimulation, the breathing rate determined for example in the ramp-down of the instructed breathing frequencies.

In an embodiment, the method comprises providing feedback: visually, or audially, or haptically or any combination thereof. These are advantageous ways in delivering information on how well the breathing stimulates the vagus nerve.

In an embodiment, in the method, in timing the breathing events to the person with the breathing pacer and with the breathing rate sequence comprising breathing rates, a successive breathing rate of the breathing rate sequence is lower than a previous breathing rate of the breathing rate sequence.

In an embodiment, in the method, in timing the breathing events to the person with the breathing pacer and with the breathing rate sequence comprising breathing rates, a successive breathing rate of the breathing rate sequence is higher than a previous breathing rate of the breathing rate sequence.

In an embodiment, in the method, in timing the breathing events to the person with the breathing pacer and with the breathing rate sequence comprising breathing rates, the breathing rate sequence is a predetermined breathing rate sequence.

Said three breathing rate sequences above are advantageous for iterating the best resonance score indicating the strongest vagus nerve stimulation for the person.

In an embodiment, the method is executed in a system according to the system aspect and its embodiments of the invention. The system defined above is an advantageous system to execute the method.

As an aspect of the present invention, a computer program is disclosed. The computer program comprises executable instructions which are configured to execute all the steps of a method according to the method and its embodiments as defined above in a computer, or a mobile computing device, or network data server, or any combination thereof. A computer program is an advantageous way to implement the method in various types of computer hardware.

The invention is based on the idea of determining and informing the person of the breathing rate from the instantaneous heart rate data that provides the best vagus nerve stimulation through breathing by altering a sequence of instructed or “timed” breathing rates for the person. Optimal vagus nerve stimulation through breathing may be achieved when the variation of the instantaneous heart rate becomes periodic, has a steady and high amplitude and alternates around a mean. By fitting a periodic biofeedback function, for example a time dependent sinusoidal function with an amplitude, angular frequency, phase and offset (mean) value, to the instantaneous heart rate data, the amplitude of the variation and the cumulative difference between the periodic biofeedback function and the instantaneous heart rate data become available very quickly when compared to prior art methods. In the optimal breathing frequency, the amplitude of the variation is maximal and the cumulative difference (that is, the error) between the fitted periodic biofeedback function and the measured data is minimal.

The invention has many advantages. The optimal breathing rate for vagus nerve stimulus through breathing can be determined without any invasive procedures or electrical stimulation. Breathing measurements in, from or by the respiratory tract are also not needed. Instead, mere heart rate measurements of individual heartbeats are sufficient. Heart rate measurements are very convenient with modern technologies. With the fitting of the periodic biofeedback function on instantaneous heart rate data, a very quick determination of the relevant parameters of the function is achieved as the data does not have to be subjected to complex spectral analyses like Fourier transforms that may require very long samples of heartbeat information to become reliable.

With the invention, information on the heart rate variation and consequently the indication on how well various breathing rates suit the vagus nerve stimulation may become available between five to ten seconds. This is considerably faster than with prior art systems or methods.

It is to be noted that the presented invention does not disclose a diagnostic or therapeutic invention. To be a diagnostic invention, there would need to be one or more “normal” ranges of heart rate variabilities, and then one or more “abnormal” ranges of heart rate variabilities, and, then according to the invention, a determination and diagnostics should be reached based on the measured heart rate variability, and the normal and abnormal ranges. However, the present invention is not aimed into such determination.

Similarly, to be a therapeutic invention, the invention (system or method) should directly have a therapeutic effect. However, it is the breathing of the person that may stimulate the vagus nerve to a varying degree, and the strength of the stimulus may be dependent on the breathing rate. However, the invention lets the user to monitor the effect of the breathing to the heart rate variability. High heart rate variability may have health benefits and it may be indicative of improved vagus nerve stimulation, achieved through “correct” breathing. Thus, the invention allows monitoring of the person, and it is up to the user to perform the breathing through the results of the monitoring. The breathing with a certain breathing rate may then have health related positive effects.

For the purposes of this text, “vagus nerve stimulation” means “vagus nerve stimulation through breathing” that may be guided and observed by the system, method and computer program, as the system, method or computer program disclosed in the present text do not, in any way, directly stimulate the body of the person.

### BRIEF DESCRIPTION OF THE DRAWINGS

The invention is described in detail by means of specific embodiments with reference to the enclosed drawings, in which

FIG. 1a shows schematically an embodiment of the system and the person being monitored,

FIGS. 1b-1d show and the timing and breathing instructions of the breathing pacer based on different breathing rate sequences,

FIG. 2 shows concepts related to an embodiment of a periodic biofeedback function,

FIG. 3a shows details of a fitting process between the periodic biofeedback function and the instantaneous heart rate data,

FIG. 3b illustrates the concept of resonance score,

FIG. 4 shows concepts related to an embodiment of a periodic biofeedback function, the sinusoidal function,

FIG. 5 shows concepts related to an embodiment of a periodic biofeedback function, a skewed sinusoidal function,

FIGS. 6a-6c shows schematically concepts related to an embodiment of the system related to a so-called resonance score, and breathing pacer,

FIG. 7 shows schematically concepts related to another embodiment of the system,

FIG. 8 shows schematically concepts related to yet another embodiment of the system,

FIG. 9 shows schematically an embodiment of the method according to the current invention,

FIGS. 10a and 10b show schematically another embodiment of the method according to the current invention, and

FIG. 11 shows schematically aspects of a computer program according to an aspect of the current invention.

### DETAILED DESCRIPTION OF THE INVENTION

In the Figures and in this text, like numbers (for example 112) and like labels (for example 112m) relate to like elements.

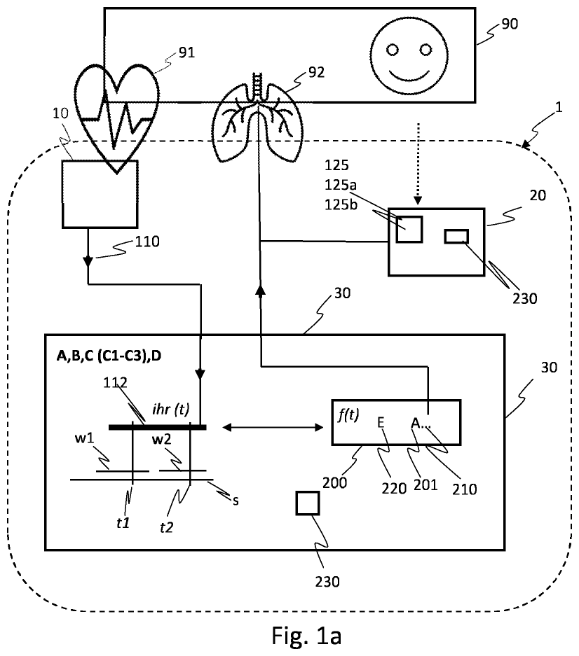

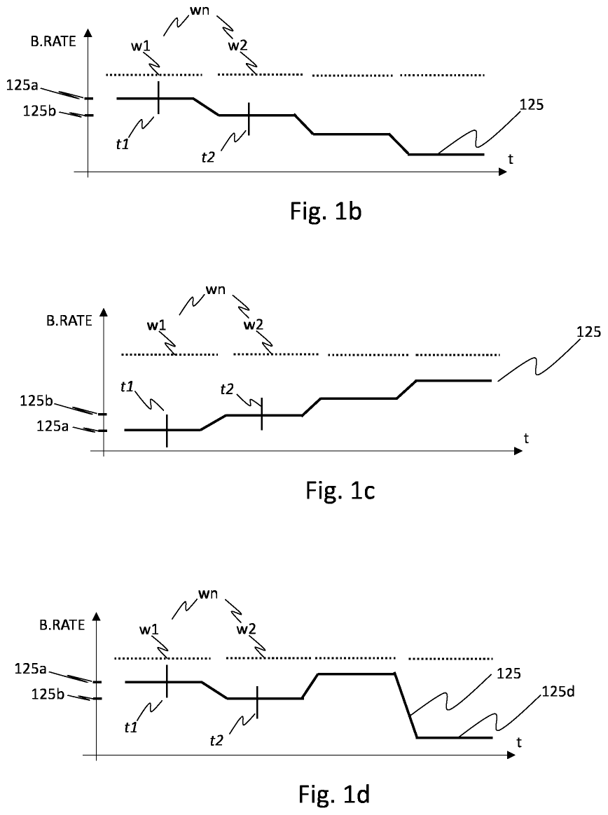

FIG. 1a shows schematically an aspect of the present invention, a system 1 for monitoring health of a person 90 during a health monitoring session. The monitoring is performed by analysing heart rate of the person 90 and determining from the heart rate the best breathing rate for vagus nerve stimulation. Determining the personal breathing rate that optimally stimulates the vagus nerve of a person is a relevant technical problem in the fitness and medical community and subject to a growing interest in the field.

For the purposes of this text, a health monitoring session, labelled s, is any period of time during which the person is interested in observing his/her physiological matters related for example to breathing, cardiac activity or state of the nervous system, and in particular the interest of the person may be in the optimal vagus nerve stimulation through breathing.

For the purposes of this text, the terms “breathing rate” and “breathing frequency” are used interchangeably, and they all relate to a time-dependent breathing frequency of a person, calculated for example from the inverse of timespan of start of two subsequent inhalations when a person breathes. Usually breathing frequency is expressed in breathes per one minute (60 seconds), and it is usually 3-8 breaths/minute for an adult person in rest, especially when the person attempts strong or optimal vagus nerve stimulation through breathing.

For the purposes of this text, “within the time window w” means a period of time that starts and ends in the time window w, but does not necessarily span the entire time window w. Letter w is the label for a time window, as are symbols w1 and w2.

For the purposes of this text, the “instantaneous heart rate” f₁ may be defined as the inverse of the interval (in time) of two consecutive heartbeats T_{R-R}, and usually this heart rate is expressed as a frequency in minutes. The interval may be measured, for example, as the interval between timepoint of two consecutive R waves in the so-called QRS complex of a beating human heart. Thus, stated exactly, f₁=60/T_{R-R}, implying that an instantaneous heart rate of 60 beats in one minute (“BPM”, beats per minute) means that the interval between two consecutive R waves of a beating heart is 1 s. Instantaneous heart rate varies from one heartbeat to the next, and this variation is called, in general, heart rate variability. Instantaneous heart rate usually varies periodically and around a mean. For example, mean of an instantaneous heart rate mean may be 70 beats in one minute, and the instantaneous heart rate may vary with an amplitude of 8 beats/minute around the mean such that minimum instantaneous heart rate is 62 beats/minute and maximum 78 beats/minute over a period of time when the person is at rest. Especially breathing, as is discussed in the present text, has the ability to alter the instantaneous heart rate.

The system 1 comprises a heart rate sensor 10 which is arranged to measure heart beats of the person 90 for providing heart rate measurement data 110. Heart of the person is indicated with symbol 91, and breathing information is associated with symbol of lungs 92. Several kinds of heart rate sensors exist, for example electrocardiographic sensors that measure the electrical activity of heart and comprise usually an elastic band or belt worn around the chest area of a person 90, and photoplethysmographic sensors that detect the heart rate based on variations of optical properties of tissue and which comprise for example a clip with a sensor worn in an earlobe or a finger of a person 90.

The system also comprises a breathing pacer 20 configured to indicate breathing information to the person 90. The breathing pacer 20 may be a device, or a utility arranged, for example, in a mobile computing device like a smartphone or a tablet computer that is configured to guide the person to adapt a breathing rate that optimizes the vagus nerve stimulation through breathing.

The breathing pacer 20 may comprise digital electronics, analogue electronics, memory or memories, power unit like a battery, ASICs, FPGAs, digital busses, microcontrollers and microprocessors to arrange its various operations. The breathing pacer 20 may also comprise one or more input devices like a keyboard, a microphone or a touch screen display, and output devices like a LED, a display, a loudspeaker or a haptic device like a linear resonant actuator.

Specifically, the breathing pacer 20 is configured to time the breathing events to the person 90 with a breathing rate sequence 125 comprising breathing rates 125a, 125b. This is illustrated in FIGS. 1b-1d, where in each of the Figures, four breathing rates are shown, two labelled as 125a and 125b.

For the purposes of this text, “timing breathing events” or “to time the breathing events” means that the breathing pacer 20 signals or is arranged to signal to the person the moment in time the person should breathe, for example, start the next inhalation.

For this purpose, the health monitoring session s comprises a time window sequence wn, the time window sequence wn comprising time windows labelled w1, w2, such that each of the breathing rates 125a,125b of the breathing rate sequence 125 is associated with associated time window w1, w2 of the time window sequence wn.

The system 1 comprises also a data processing system 30 which is configured (as operation A) to receive the heart rate measurement data 110 from the heart rate sensor 10 during the health monitoring session s. This data may be transmitted for example in digital or analogue voltage signals for example with electrical connections from the heart rate sensor 10 to the data processing system 30. The data processing system 30 which is also configured (as operation B) during the health monitoring session s to arrange the breathing pacer 20 to time the breathing events to the person 90 in each of the breathing rates 125a, 125b.

The data processing system 30 is also (as operation C) configured to analyse the heart rate measurement data 110.

In the analysis (operation C), the data processing system 30 is configured to, (as operation C1) calculate instantaneous heart rate data 112 comprising the instantaneous heart rate of each measured heartbeat. The instantaneous heart rate f₁ 112 may be defined as the inverse of the interval (in time) of two consecutive heartbeats, as defined already above. The calculating is performed over each of the time windows w1, w2 in the health monitoring session s, each of the time windows w1, w2 comprising a timepoint t1, t2. The instantaneous heart rate data 112 is calculated based on the heart rate measurement data 110 received from the heart rate sensor 10. As an example, the time window w1 might start at timepoint 1500s (measured from an arbitrary point in time) and end at timepoint 1540s, (making the time window 40s wide) and timepoint t1 can be the midmost timepoint at 1520s. In a practical implementation, the time windows w1, w2 may comprise discrete timepoint, for example 10, 20 or 40 discrete timepoint where the instantaneous heart rate data 112 is represented for example by digital computing means. Thus, the heath monitoring session s comprises many, possibly overlapping time windows w1, w2. The time windows w1, w2 may be consecutive in time, arranged in the time window sequence wn.

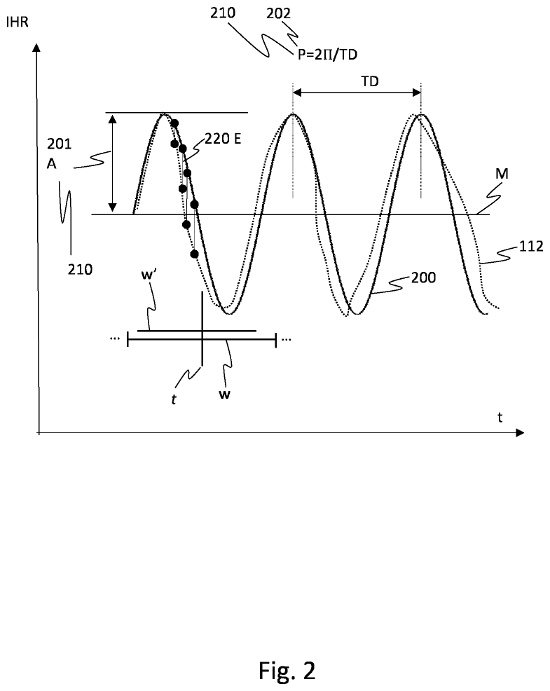

Next, referring to FIG. 2, in the analysis, the data processing system 30 (as operation C2) is configured to provide a periodic biofeedback function 200, which comprises parameters 210. The parameters 210 comprise an amplitude A 201 of the periodic biofeedback function 200. In FIG. 2, the periodic biofeedback function is shown with solid line 200, and the instantaneous heart rate data 112 as the dotted line over a time axis t. The periodic biofeedback function 200 comprises also a cumulative difference E 220 relative to the instantaneous heart rate data 112. In FIG. 2, the cumulative difference E 220 may be the difference between the curves 200 and 112 in an arbitrary time interval w′ within the time window w which may be time window w1, w2 etc. The arbitrary time interval w′ may be within the time window w completely or partially. As shown in FIG. 2, the instantaneous heart rate data 112 (IHR as the y axis) approximately oscillates around an approximate mean M, with an approximate amplitude A and with an approximate angular frequency P. The values for the parameters A, P and M may be found by arranging the data processing system 30 to the fit the periodic biofeedback function 200 to the instantaneous heart rate data 112. Mean M may be for example 65 beats per minute (BPM), and the amplitude A may be 5 BPM.

In the analysis, the data processing system 30 (as operation C3) is also configured to fit the periodic biofeedback function 200 into the instantaneous heart rate data 112 within each of the time windows w1, w2 to determine the parameters 210 of the periodic biofeedback function 200 at each of the timepoints t1, t2, and to determine the cumulative difference E 220 at each of the timepoints t1, t2.

The data processing system 30 is also configured, as operation D, to store the parameters 210 of the periodic biofeedback function 200 at each of the timepoints t1, t2, and the cumulative difference E 220 at each of the timepoints t1, t2. Storing is arranged, for example, for determining the best breathing frequency for vagus nerve stimulation after the breathing pacer suggests and times different breathing frequencies 125a, 125b of the breathing rate sequence 125. In other words, storing may be arranged for determining the best breathing frequency for vagus nerve stimulation after the breathing rates 125a, 125b of the breathing rate sequence 125 are tried by the person 90.

The data processing system 30 may also be configured, in operation D, to store the breathing rates 125a, 125b associated with the time windows w1, w2 comprising the timepoints t1, t2.

The data processing system 30 may also be configured provide the time window sequence wn such that the time window sequence wn comprises time windows w1, w2.

The data processing system 30 may also be configured to provide the breathing rate sequence 125 comprising the breathing rates 125a,125b such that each of the breathing rates 125a, 125b of the breathing rate sequence 125 is associated with the associated time window w1, w2 of the time window sequence wn.

Thus, with the system 1, determining the optimal vagus nerve stimulation is achieved with no complex and inconvenient measurements for example in the respiratory tract, and also quickly, as long sample periods needed by a Fourier transform are not needed.

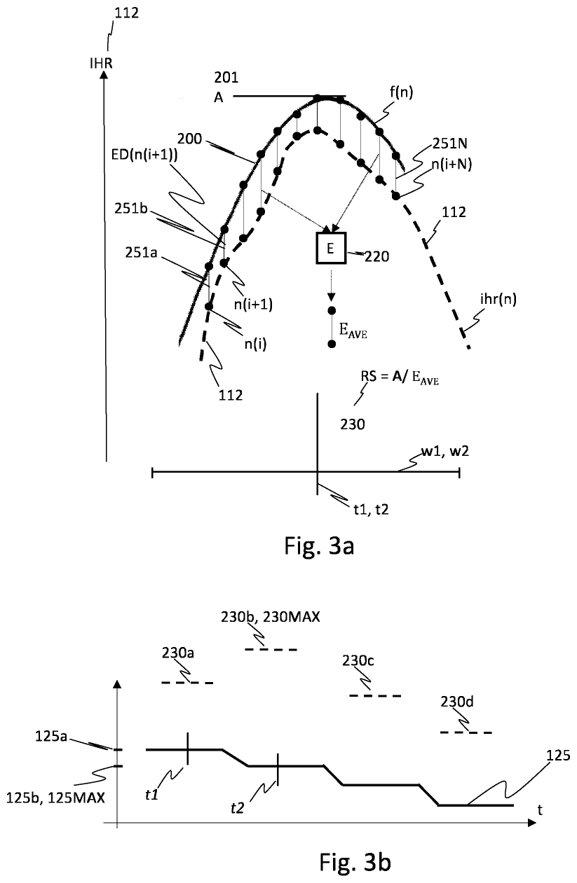

In an embodiment, referring to FIG. 3a, in the fitting (the operation C3), the data processing system 30 is configured to determine the cumulative difference E, indicated with symbol 220 between the instantaneous heart rate data 112 and the periodic biofeedback function 200 within each of the time windows w1, w2, the time windows w1, w2 comprising the timepoints t1, t2. The data processing system 30 is also configured to perform the fitting (operation C3) based on a calculated minimum of the cumulative difference E 220 within each of the time windows w1, w2 to determine the parameters 210 of the periodic biofeedback function 200 at each of the timepoints t1, t2, the time windows w1, w2 comprising the timepoints t1, t2.

After the fitting (the operation C3), the data processing system 30 may also be configured determine the cumulative difference E 220 at each of the timepoints t1, t2 from the calculated minimum of the cumulative difference E 220 within each of the time windows w1, w2.

After the fitting (the operation C3), the data processing system 30 may also be configured to set the value of the cumulative difference E 220 at each of the timepoints t1, t2 to the value of the calculated minimum of the cumulative difference E 220 within each of the time windows w1, w2. In other words, after the fitting, the cumulative difference E 220 may have a minimum value within each of the time windows w1, w2.

The concept of the cumulative difference E is illustrated further in FIG. 3a, where the instantaneous heart rate data 112 is show as a curve 112. Instantaneous heart rate data 112 comprises discrete instantaneous heart rate values at discrete points in time (three discrete points shown, n(i), n(i+1), and n(i+N)) within the time window w (which may be for example w1, w2), the time window w comprising the timepoint t (which may be for example t1, t2). The discrete points in time may comprise the timepoint t. For each discrete point in time, a discrete difference ED (three discrete differences labelled in FIG. 3a, marked with 251a, 251b, 251N) is determined that contributes to the cumulative difference E 220. The discrete differences (251a, 251b, 251N) may be determined as the subtraction of the values of the instantaneous heart rate data 112 (represented by function ihr(n)) at the corresponding discrete timepoint n(i) . . . n(i+N), and the values of the periodic biofeedback function 200 f(n) in the discrete timepoint n(i) . . . n(i+N), where . . . denotes “from n(i) to n(i+N)”. In other words, ED(n)=ihr(n)−f(n).

In an embodiment, to provide the cumulative difference E 220, the data processing system 30 may be arranged to calculate the cumulative difference E 220 as the sum of absolute values of the discrete differences (251a-251N) between values of the instantaneous heart rate data 112 and values of the periodic biofeedback function 200 at the discrete timepoints n(i) . . . n(i+N),

$$E ( t ) = {\sum }_{j = i}^{N + i} \vert ihr ( n ( j ) ) - f ( n ( j ) ) \vert$$

where the | | denotes the absolute value and t is a timepoint within the discrete datapoints n(j).

As another embodiment, the data processing system 30 may be arranged to calculate the cumulative difference E 220 as the sum of squared values of the discrete differences (251a-251N) between values of the instantaneous heart rate data 112 and values of the periodic biofeedback function 200, wherein t is a timepoint within the discrete datapoints t=n(j),

$$E ( t ) = {\sum }_{j = i}^{N + i} {( ihr ( n ( j ) ) - f ( n ( j ) ) )}^{2} .$$

In fitting, in operation C3, the data processing system 30 may be arranged to vary the parameters 210 of the periodic biofeedback function 200, and for each set of parameters 210, determine the cumulative difference E 220. In fitting, in operation C3, the data processing system 30 may be also arranged determine the calculated minimum of the cumulative difference E 220, and the parameters 210 of periodic biofeedback function 200 used to reach the calculated minimum of the cumulative difference 220. To determine the calculated minimum of the cumulative difference E 220, the data processing system 30 may be arranged to vary of the parameters 210 of the periodic biofeedback function 200 for a number of iteration loops. The data processing system 30 is arranged to base the variation of the parameters to one or more search algorithms or fitting methods. The data processing system 30 is then configured to determine the parameters 210 resulting in a calculated minimum of the cumulative difference 220, as the parameters 210 resulting in a calculated minimum of the cumulative difference 220 are then the result of the fitting, providing the values of the parameters 210 of the periodic biofeedback function 200.

In an embodiment, the data processing system 30 is configured to perform the fitting (in operation C3) within each the time windows w1, w2 with a least squares method, or a modified least squares method, or a random search, or an exhaustive search, or any combination thereof. In other words, the data processing system 30 may be arranged to base the varying of the parameters 210 of the periodic biofeedback function 200 within each the time windows w1, w2 to determine the calculated minimum of the cumulative difference E 220 on various search algorithms or fitting methods like a least squares method, a modified least squares method, a random search method or an exhaustive search method. The data processing system 30 may be also arranged to use any combination of the search algorithms or fitting methods of the least squares method, the modified least squares method, the random search method or the exhaustive search method to determine the calculated minimum of the cumulative difference E 220.

With the random search method or fitting method, the data processing system 30 is configured to perform the fitting (operation C3) by varying the parameters 210 of the periodic biofeedback function 200 within each the time windows w1, w2 randomly such that the values of the parameters 210, within ranges of feasible values, are arranged to be randomly assigned during the iteration loops. The data processing system 30 is then arranged to choose the set of parameters 210 reaching the calculated minimum of the cumulative difference 220 as the parameters 210 of the periodic biofeedback function 200. The data processing system 30 may also be arranged to start the random assignment of the parameters from an initial guess of one or more of the parameters.

With the exhaustive search method or fitting method, the data processing system 30 is configured to perform the fitting (operation C3) by vary the parameters 210 of the periodic biofeedback function 200 within each the time windows w1, w2 such that every combination of the parameters 210, within ranges of feasible values in a discrete set of the feasible values, are arranged to be tried during the iteration loops. For example, it may be determined that the maximum amplitude A 201 of the instantaneous heart rate is 20 beats/minute, all values of A, from zero to 20 beats/minute are tried with a discrete interval of for example 0.2 beats/minute. In other words, values 0, 0.2, 0.4, . . . 20 are tried when the amplitude parameter A 201 is varied. The same holds of other parameters. The data processing system 30 is then arranged to choose the set of parameters 210 achieving the calculated minimum of the cumulative difference 220 as the parameters 210 of the periodic biofeedback function 200. The data processing system 30 may also be arranged to start the variation of the parameters 210 from an initial guess of one or more of the parameters.

With the least squares search method or fitting method, the data processing system 30 is arranged to perform the fitting (operation C3) by varying the parameters 210 of the periodic biofeedback function 200 within each the time windows w1, w2 based on the numerical Gauss-Newton algorithm to find the calculated minimum of the cumulative difference E 220. By representing the parameters 210 of the periodic biofeedback function with a vector β, and discrete difference between the periodic biofeedback function 201 and the instantaneous heart rate data 112 at discrete timepoint n(j) by a “residual” rⱼ as function of the parameters β 201 (number of parameters being k), calculated minimum of the cumulative difference E 220 is found when sum of squares S(β) is minimized, where

$$S ( β ) = {\sum }_{j = i}^{N + i} {{r}_{j} ( β )}^{2} .$$

With an initial guess for the parameters 210 of #°, the data processing system 30 is arranged to vary the parameters 210 based on an iteration rule that may be for example

$${β}^{L + 1} = {β}^{L} - {( {J}_{r}^{T} {J}_{r} )}^{- 1} {J}_{r}^{T} r ( {β}^{L} ) ,$$

where L is the Lth iteration loop, and J is the Jacobian matrix with elements,

$${( {J}_{r} )}_{jk} = \frac{\partial {r}_{j} ( {β}^{L} )}{\partial {β}_{k}} ,$$

T denotes the matrix transpose and superscript −1 the matrix inverse.

With the modified least squares search method or fitting method, the data processing system 30 is arranged to perform the fitting (operation C3) by varying the parameters 210 of the periodic biofeedback function 200 within each the time windows w1, w2 based on the numerical Levenberg-Marquardt algorithm to find the calculated minimum of the cumulative difference E 220. The Levenberg-Marquardt algorithm is similar to the Gauss-Newton algorithm, but with the Levenberg-Marquardt algorithm the iteration rule may be for example

$${β}^{L + 1} = {β}^{L} - {( {J}_{r}^{T} {J}_{r} - λ I )}^{- 1} {J}_{r}^{T} r ( {β}^{L} ) ,$$

where λ is a damping parameter and I is an identity matrix. The damping factor λ may be arranged by the data processing system 30 for the iteration loops to a value, based on the error of the iteration, determined by the difference of the results of two consecutive iteration loops, that configures the iteration to find the parameters 210 of the periodic biofeedback function 200 faster. If the error of the iteration increases, the damping factor may be increased. If the error of the iteration decreases, the damping factor may be decreased. Clearly, with λ=0, the Levenberg-Marquardt algorithm reduces to the Gauss-Newton algorithm.

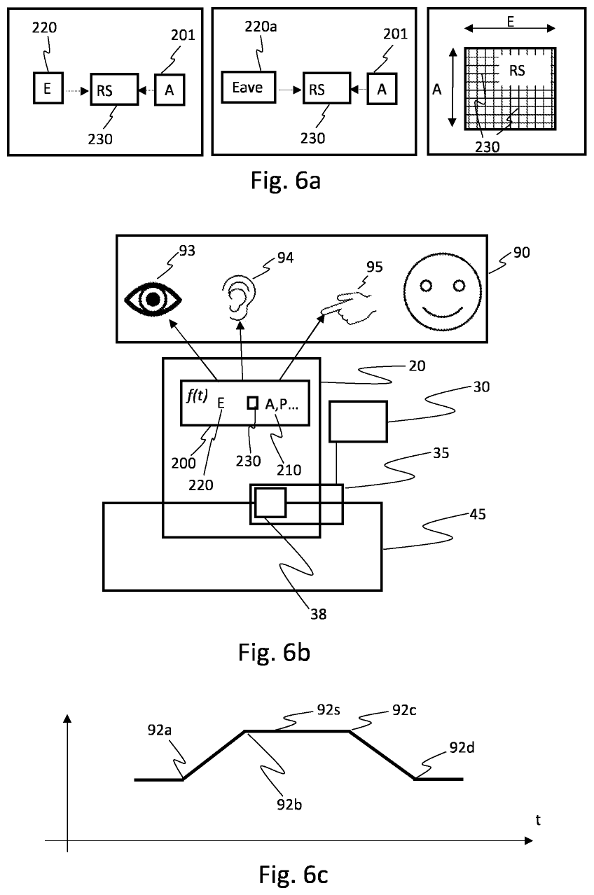

FIG. 3a together with FIGS. 3b and 6a illustrate a concept of resonance score 230. Optimal vagus nerve stimulation is achieved when the behaviour of the instantaneous heart rate data 112 is periodic and alternates over time around a mean with a large amplitude A 201. As the instantaneous heart rate data 112 is arranged to be represented by the periodic biofeedback function 200 in the data processing system 30, the cumulative difference E 220 between the periodic biofeedback function 200 and the instantaneous heart rate data 112 may also determine the impact on the vagus nerve stimulation, as the amplitude A 201 may also be large when the periodic biofeedback function 200 does not follow the instantaneous heart rate data 112 well. Thus, as a measure of the ability of the breathing to stimulate vagus nerve, the data processing system 30 may be configured to determine a resonance score 230 based on the amplitude 201 of the periodic biofeedback function, and on the cumulative difference E 220.

As indicated in FIG. 3b, the data processing system 30 may be configured to determine a resonance score 230 (four resonance scores, 230a-230d, are shown) based on the amplitude 201 of the periodic biofeedback function, and on the cumulative difference E 220 such that the determined resonance score 230 indicates maximal vagus nerve stimulation 230MAX. The data processing system 30 may also be configured to determine the breathing rate 125MAX associated with the resonance score 230 indicating the maximal vagus nerve stimulation 230MAX.

For example, in reference to FIG. 6a, the resonance score RS 230 may be defined as a ratio of the amplitude 201 of the periodic biofeedback function, and an average difference EAVE of the cumulative difference E 220 of N discrete timepoint within the time window w, which may be w1, w2 etc: E_{AVE}=E/N. In other words, RS=A/E_{AVE}.

In reference to FIG. 6a, the resonance score 230 may also be defined by a two-dimensional table of values for amplitude 201 and cumulative difference 220 E. The resonance score value RS 230 may be then pre-recorded for the A and E entries in the table, for example in the data processing system 30. Generalizing, an arrangement for the determination of the resonance score 230 that indicates success in the vagus nerve stimulation based on a large value of amplitude A 201 and low value of cumulative difference E 220 is possible.

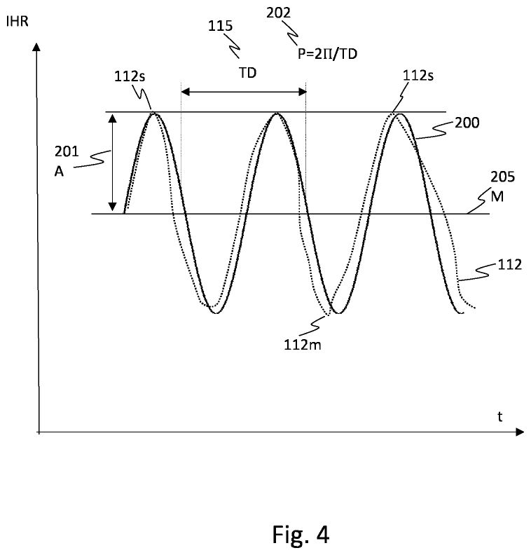

Turning to FIG. 4, in an embodiment, in fitting and in determining the parameter indicating the amplitude A 201 of the periodic biofeedback function 200 at each of the timepoints t1, t2, the data processing system 30 is arranged to determine a minimum instantaneous heart rate 112m and a maximum instantaneous heart rate 112s of the instantaneous heart rate data 112 within each of the time windows w1, w2, subtract the minimum instantaneous heart rate 112m from the maximum instantaneous heart rate 112s, divide the value of subtraction by 2, and determine the parameter indicating the amplitude A 201 of the periodic biofeedback function 200 at each of the timepoints t1, t2 based on the value of the division. The data processing system 30 may be arranged to set the amplitude parameter A 201 to value of the division directly such that in the determination of the parameters 210, in fitting, the data processing system 30 keeps parameter indicating the amplitude A 201 of the periodic biofeedback function 200 at a value based on the value of the division. Alternatively or additionally, the data processing system 30 may use the value of the division as an initial guess in the search algorithm or fitting method.

Still referring to FIG. 4, in an embodiment, the parameters 201 of the periodic biofeedback function 200 comprise an angular frequency P 202, and in fitting (operation C3) and in determining the parameter indicating the angular frequency P 202 of the periodic biofeedback function 200 at each of the timepoints t1,t2, the data processing system 30 is arranged to determine a cycle time TD 115 of the instantaneous heart rate data 112 within each of the time windows w1, w2, and determine the parameter indicating the angular frequency P 202 of the periodic biofeedback function 200 each of the timepoints t1, t2 based on the cycle time TD 115. As shown in FIG. 4, the data processing system 30 may be arranged to determine two subsequent, essentially same values (or two values that are within a margin of error) within the instantaneous heart rate data 112 and then measure the cycle time TD between two subsequent, essentially same values, and then determine the angular frequency from a known relation P=2Π/TD. The data processing system 30 may be arranged to determine the two subsequent, essentially same values (or two values that are within a margin of error) on two successive rising or falling edges of the instantaneous heart rate data 112. The data processing system 30 may be also arranged to determine the cycle time TD 115 between two successive maximum or two successive minimum values of the instantaneous heart rate data 112. The data processing system 30 may be arranged to set the angular frequency parameter P 202 to value based on cycle time TD 115 directly such that for the determination of the parameters 210, in fitting, the data processing system 30 keeps parameter indicating the angular frequency P 202 of the periodic biofeedback function 200 directly based on the cycle time TD 115. Alternatively or additionally, the data processing system 30 may use the value based on the cycle time TD as an initial guess in the search algorithm or fitting method.

The angular frequency P 202 may also indicate the measured breathing rate f_{B} 120 of the person 90 through relation f_{B}=P/2Π. The breathing pacer 20 may be arranged to show the measured breathing rate 120, for example to the person 90.

The breathing pacer 20 may be arranged to show the measured breathing rate 120 and at least one (instructed) breathing rate 125a of the breathing rate sequence 125.

The breathing pacer 20 may be arranged to show information based on a difference between the measured breathing rate 120 and at least one (instructed) breathing rate 125a of the breathing rate sequence 125.

Still referring to FIG. 4, in an embodiment, the parameters 210 of the periodic biofeedback function 200 comprise a mean M 205 of the periodic biofeedback function 200. In determining the parameter indicating the mean M 205 of the periodic biofeedback function 200 at the timepoint t, the data processing system 30 is arranged to determine a mean value of the instantaneous heart rate data 112 within each of the time windows w1, w2, and determine the parameter indicating the mean M 205 of the periodic biofeedback function 200 at each of the timepoints t1, t2 based on the mean value. The data processing system 30 may be arranged to set the mean M 205 to the mean value directly such that for the determination of the parameters 210, in fitting, the data processing system 30 keeps the mean M 205 of the periodic biofeedback function 200 based on the mean value. Alternatively or additionally, the data processing system 30 may use the mean value an initial guess in in the search algorithm.

The amplitude A 201 of the instantaneous heart rate data 112 indicates or determines the heart rate variability of the person 90 in frequency domain (1/time unit) and is thus a frequency domain characterisation. A measure of the heart rate variability in time domain may also be calculated as (1/(M−A))−(1/(M+A))=2A/(M²−A²)≈2A/M², where A is the amplitude A 201, and M the mean 205.

In an embodiment, and still referring to FIG. 4, the periodic biofeedback function f(t) 200 at each of the timepoints t1,t2 comprises a sinusoidal function sin( ) such that f(t) is defined as f(t)=A sin(Pt−T)+M, in which A is the amplitude A 201 of the instantaneous heart rate data 112 within each of the time windows w1, w2, P is the angular frequency P 202 of the instantaneous heart rate data 112 within each of the time windows w1, w2, T is a time displacement, and M is a mean value M 205 of the instantaneous heart rate data 112 within each of the time windows w1, w2. The data processing system 30 may be configured to use the sinusoidal function sin( ) and fit it to the instantaneous heart rate data 112 within each of the time windows w1, w2 to determine the parameters 210 of the periodic biofeedback function 200 at each of the timepoints t1, t2. Through fitting, the parameters A, P and M are determined for the timepoint t1, t2 of each of the associated time windows w1, w2.

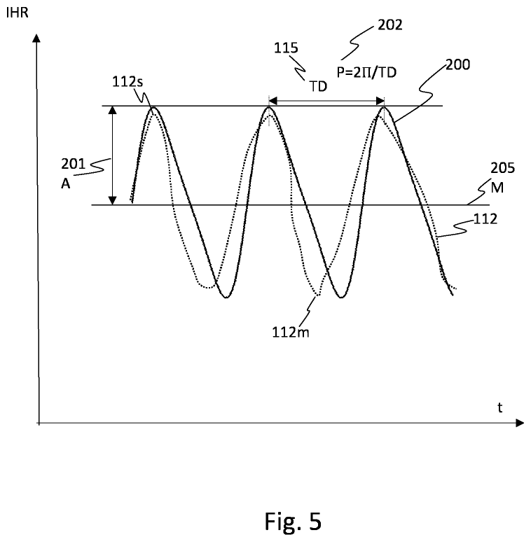

In an embodiment, and referring next to FIG. 5, the periodic biofeedback function f(t) 200 at each of the timepoints t1, t2 comprises a skewed sinusoidal function sk sin( ) such that f(t) is defined as f(t)=A sk sin(Pt−T)+M=A sin[(Pt−T)+k*sin(Pt−T)]+M, in which A is the amplitude A 201 of the instantaneous heart rate data 112 within each of the time windows w1, w2, P is the angular frequency P 202 of the instantaneous heart rate data 112 within each of the time windows w1, w2, T is a time displacement, k is a skew factor, and M is a mean value M 205 of the instantaneous heart rate data 112 within each of the time windows w1, w2. The asterisk denotes multiplication. The skew factor k can be set to configure the periodic biofeedback function's 200 waveform to be asymmetric so that rise time (from zero value to the positive or negative amplitude value or peak value) is shorter in time than the fall time from the amplitude value back to the zero value. The skew factor may be 0-1, more advantageously 0.3-0.6 or most advantageously 0.4-0.5. A skewed sinusoidal function is advantageous as the inhalation is often somewhat shorter than the exhalation and thus the asymmetric waveform may easier be fit to the instantaneous heart rate data 112 than a symmetric waveform, symmetry defined relative to the vertical axis of once cycle of the periodic biofeedback function, when said vertical axis located to the timepoint of maximum or minimum value of the function. Through fitting, the parameters A, P and M are determined for the timepoint t1, t2 of each of the associated time windows w1, w2.

In an embodiment, the periodic biofeedback function 200 at each of the timepoints t1, t2 is an alternating trapezoidal pulse train function with an amplitude A, angular frequency P and mean value of M.

In an embodiment, that the data processing system 30 is configured to analyse (as operation C) the heart rate measurement data 110 for each heartbeat measured with the heart rate sensor 10.

In another embodiment, the data processing system 30 is configured to analyse, (as operation C), the heart rate measurement data 110 for every second heartbeat measured with the heart rate sensor 10.

In another embodiment, the data processing system 30 is configured to analyse, (as operation C), the heart rate measurement data 110 such that the frequency of analysis is based on the cumulative difference E 220 such that increasing cumulative difference E 220 makes the frequency of analysis higher, maximally every heartbeat measured with the heart rate sensor 10. Decreasing cumulative difference E 220 makes the frequency of analysis lower such that minimally the data processing system 30 is configured to analyse, as operation C, the heart rate measurement data 110 every fifth, every tenth of every fifteenth heartbeat measured with the heart rate sensor 10.

In an embodiment, referring to FIG. 6b, the breathing pacer 20 is configured to provide feedback to the person 90 within at least one of the time windows w1, w2 based on the parameters 210 of the periodic biofeedback function 200; or the cumulative difference E 220; or any combination thereof. The feedback may be for example indication of the amplitude A 201 of the periodic biofeedback function 200. High amplitude implies strong vagus nerve stimulation. Similarly, the feedback may be for example indication of the cumulative difference E 220. Good fit of the periodic biofeedback function 200 to the instantaneous heart rate data 112, indicated by a low cumulative difference E 220, is also an indication of strong vagus nerve stimulation. In particular, a high amplitude A 201 and a low cumulative difference E 220 is an indication of a strong vagus nerve stimulation.

Optimal vagus nerve stimulation is achieved when the behaviour of the instantaneous heart rate data 112 is periodic and alternates over time around a mean with a large amplitude A 201. As the instantaneous heart rate data 112 is arranged to be represented by the periodic biofeedback function 200 in the data processing system 30, the cumulative difference E 220 between the periodic biofeedback function 200 and the instantaneous heart rate data 112 may also determine the impact on the vagus nerve stimulation, as the amplitude A 201 may also be large when the periodic biofeedback function 200 does not follow the instantaneous heart rate data 112 well. Thus, as a measure of the ability of the breathing to stimulate the vagus nerve, the data processing system 30 may be configured to determine a resonance score 230. As discussed in relation to FIG. 3a, and also referring to FIG. 6a, in an embodiment, data processing system 30 of system 1 is arranged to determine the resonance score 230 based on the amplitude A 201 of the periodic biofeedback function 200 and the cumulative difference E 220 at least within one of the time windows w1, w2. The breathing pacer 20 is also configured to provide feedback to the person 90 within at least one of the time windows w1, w2 based on the resonance score 230.

In an embodiment, the data processing system 30 is configured to determine a resonance score 230 from the amplitude A 201 of the periodic biofeedback function 200 and from the cumulative difference E 220 within each of the time windows w1, w2, and the breathing pacer 20 is configured to provide feedback to the person 90 by indicating to the person 90 the breathing rate 125a, 125b having the best resonance score 230. This is illustrated in FIG. 3b, having four resonance scores 230a-230d with the second, 230b, being the best, here highest, 230MAX. The associated breathing rate 125b is the breathing rate 125MAX with the strongest vagus nerve stimulation.

For the purposes of present text, the best resonance score 230 relates to the strongest ability of the associated breathing rate 125a, 125b to stimulate the vagus nerve.

Referring still to FIGS. 3a and 6a, in an embodiment, the data processing system 30 of system 1 is arranged to determine the resonance score 230 based on the division of the amplitude A 201 of the periodic biofeedback function 200 by an average cumulative difference E_{AVE} 220a within each of the time windows w1, w2. In other words, the resonance score RS 230 may be defined as a ratio of the amplitude 201 of the periodic biofeedback function, and an average difference E_{AVE} of the cumulative difference E 220 of N discrete timepoint within the time window w, E_{AVE}=E/N. In other words, RS=A/E_{AVE}.

The resonance score 230 may also be defined by a two-dimensional table of values for amplitude 201 and cumulative difference 220 E. Referring still to FIGS. 3a and 6a, in an embodiment, the data processing system 30 of system 1 is arranged to determine the resonance score 230 based on a tabulated and predetermined set of values for resonance score 230, the tabulated and predetermined set of values for resonance score 230 arranged based on the amplitude A 201 of the periodic biofeedback function 200 and cumulative difference E 220 within each of the time windows w1, w2. Thus, the resonance score value RS 230 may be pre-recorded or configured for the A and E entries in a table, for example in the data processing system 30, and the data processing system 30 may be arranged to look up the value for the resonance score 230 within each of the time windows w1, w2 from the table based on the amplitude A 201 of the periodic biofeedback function 200 within each of the time windows w1, w2, and cumulative difference E 220 within each of the time windows w1,w2.

Generalizing, any arrangement for the determination of the resonance score 230 that indicates success in the vagus nerve stimulation based on a large value of amplitude A 201 and low value of cumulative difference E 220, and wise versa, is possible.

Referring to FIG. 6b, the breathing pacer 20 is configured to provide feedback to the person 90 within at least one of the time windows w1, w2 visually, as indicated with symbol 93. The breathing pacer 20 may comprise a display, for example an LCD display, or a lamp or light emitting diode (LED), and visual indication may comprise for example displaying the breathing frequency in which the person is to breathe in the display, or the determination of the moment of controlled breathing with a flashing symbol on the display, or in the lamp or on the LED. For example, the breathing pacer 20 may flash a LED at the moment of time the person 90 is to begin an inhalation. The breathing pacer 20 may also show a symbol on a display momentarily, at the moment of time the person 90 is to begin an inhalation.

Referring to FIG. 6b, the breathing pacer 20 is configured to provide feedback to the person 90 within at least one of the time windows w1, w2 audially, as indicated with a symbol 94. For example, the breathing pacer 20 may comprise a loudspeaker and the breathing pacer 20 may be configured to emit an audio signal for each breath the person is to take in a controlled way, or to play a certain tune based on if the breathing frequency should be increased or decreased by the person.

Referring to FIG. 6b, Referring to FIG. 6b, the breathing pacer 20 is configured to provide feedback to the person 90 within at least one of the time windows w1, w2 haptically, as indicated with a symbol 95. For example, the breathing pacer 20 may comprise a linear resonant actuator (LRA), and the breathing pacer 20 may be configured to emit a haptic vibration signal, for example be configured to generate vibrations at the moment of time when each of the inhalations should start.

Referring still to FIG. 6b, the breathing pacer 20 is configured to provide feedback to the person 90 within at least one of the time windows w1, w2 with any combination of audial, haptic or visual feedback, for example by providing a readout of the instructed breathing frequency, flash a symbol when an inhalation is to be taken, emit a beep as an audial signal when a breath is to be taken and also emit a haptic buzz for a breath to take.

Still referring to FIG. 6b, in an embodiment, in the system 1, the breathing pacer 20 comprises third software means 35 executable on a mobile computing device 45. The third software means 35 are functionally connectable with the data processing system 30, and the third software means 35 comprises computer-executable instructions 38 for providing feedback based on the parameters 210 of the periodic biofeedback function 200 or the cumulative difference E 220 or any combination thereof. The feedback may be provided within at least one of the time windows w1, w2. Thus, the functionality of the breathing pacer 20 may be implemented as an application or “app” in a mobile computing device 45, like a smartphone or a tablet computer. The mobile computing device 45 may comprise a display, a loudspeaker and a linear resonant actuator for implementing the visual, audial and haptic feedback. A display, a loudspeaker and a haptic feedback device are provided in a modern mobile phone terminal, smartphone or a tablet computer.

A “breathing cycle” means a respiratory cycle in the present text. One breathing cycle is one sequence of inhalation and exhalation. During the health monitoring session s, the person takes one or more breaths, for example 10, 30, 50 or 70 breaths, one breath corresponding to one breathing cycle.

In an embodiment, and referring to FIG. 6c, in the system 1, the breathing pacer 20 is arranged to time the breathing events to the person 90 by indicating to the person 90 a start 92a of an inhalation of each breathing cycle. FIG. 6c shows schematically the volume of the lungs (vertical y-axis) of the person 90 during one breathing cycle, over time (horizontal x-axis), comprising start of an inhalation 92a, end of an inhalation 92b, start of an exhalation 92c and end of an exhalation 92d.

In an embodiment, and referring to FIG. 6c, in the system 1, the breathing pacer 20 is arranged to time the breathing events to the person 90 by indicating to the person 90 an end 92b of an inhalation of each breathing cycle.

In an embodiment, and referring to FIG. 6c, in the system 1, the breathing pacer 20 is arranged to time the breathing events to the person 90 by indicating to the person 90 a start 92c of an exhalation of each breathing cycle.

In an embodiment, and referring to FIG. 6c, in the system 1, the breathing pacer 20 is arranged to time the breathing events to the person 90 by indicating to the person 90 an end 92d of an exhalation of each breathing cycle.

In an embodiment, and still referring to FIG. 6c, in the system 1, the breathing pacer 20 is arranged to time the breathing events to the person 90 by indicating to the person 90 any combination of the start of an inhalation 92a, the end of an inhalation 92b, the start of an exhalation 92c and the end of an exhalation 92d. A good control of breathing is achieved, for example, by indicating to the person the start of the inhalation 92a and the start of the exhalation 92c.

In an embodiment, and still referring to FIG. 6c, in the system 1, the breathing pacer 20 is arranged to time the breathing events to the person 90 by indicating to the person 90 a same moment of the breathing cycle 92s for every breathing cycle timed by the breathing pacer 20.

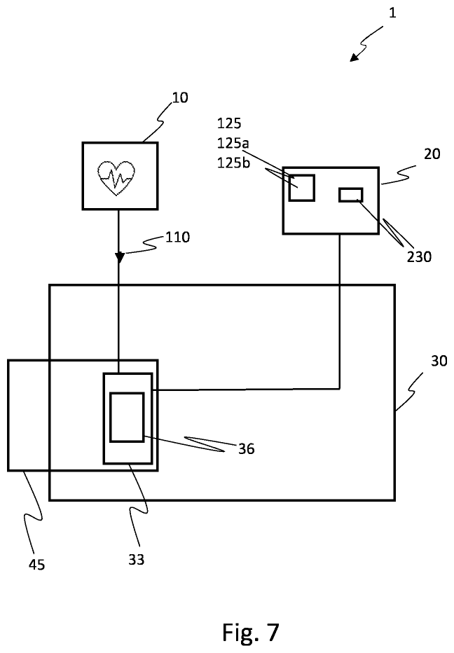

Next referring to FIG. 7, in an embodiment, in the system 1, the data processing system 30 comprises first software means 33 executable on a mobile computing device 45, the first software means 33 being functionally connectable with the heart rate sensor 10, and the first software means 33 comprises computer-executable instructions 36 to receive A heart rate measurement data, B arrange the breathing pacer 20 to time the breathing events, analyse C heart rate measurement data, and D store the parameters 210 and the cumulative difference E 220. Thus, the data processing system 30 may be arranged to be implemented through software means for example in a smartphone or a tablet computer.

The first software means 33 may also comprise computer-executable instructions 36 to store, in operation D, the breathing rates 125a, 125b associated with the time windows w1, w2 comprising the timepoints t1, t2.

As the breathing pacer 20 and the data processing system 30 may be implemented in the same unit, for example a mobile computing device 45, the breathing pacer 20 and the data processing system 30 may be functionally connected through the digital data processing units like memories, information busses, microcontrollers and microprocessors of the mobile computing device 45.

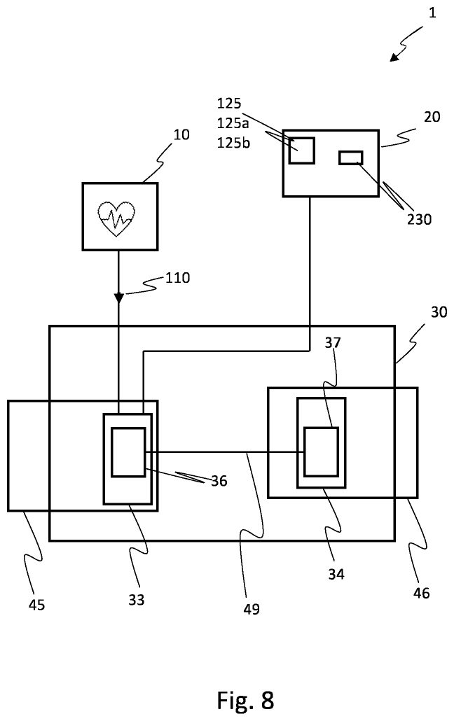

Next referring to FIG. 8, in an embodiment, in the system 1, the data processing system 30 comprises second software means 34 executable on a network data server 46, the first software means 33 and the second software means 34 are configured to exchange data over a network connection 49, and the second software means 34 comprises computer-executable instructions 37 to receive A heart rate measurement data, B arrange the breathing pacer 20 to time the breathing events, analyse C heart rate measurement data, and D store the parameters 210 and the cumulative difference E 220. The network connection 49 may comprise physical conductors or a radio interface or both, and it may be arranged through one or more of the standards of ethernet, wireless LAN, Bluetooth, GSM, 3GPP or other cable-based or wireless standard. The network data server 46 may be a server cluster of computers, or an internet cloud network computer center.

The second software means 34 may also comprise computer-executable instructions 37 to store, in operation D, the breathing rates 125a, 125b associated with the time windows w1, w2 comprising the timepoints t1, t2.

In an embodiment, in the system 1, the second software means 34 comprises computer-executable instructions 37 for displaying information based on the parameters 210 of the periodic biofeedback function 200 and the cumulative difference E 220. This is advantageous for example if the health monitoring results are to be published for example over the internet or in an app of a smartphone sharing information over the internet.

In an embodiment, referring back to FIG. 1b, the breathing pacer 20 is configured to time the breathing events to the person 90 with the breathing rate sequence 125 comprising breathing rates 125a, 125b such that a successive breathing rate 125b of the breathing rate sequence 125 is lower than a previous breathing rate 125a of the breathing rate sequence 125. In FIG. 1b, breathing rate 125b is lower, that is, less, than breathing rate 125a. Thus, a ramp-down of instructed breathing rates (ramp-down in time) is provided in the breathing rate sequence 125. There may be for example seven different breathing rates in a breathing rate sequence, 7 breaths/minute (7 BRPM), 6.5 BRPM, 6 BRPM, 5.5 BRPM, 5 BRPM, 4.5 BRPM and 4 BRPM in the breathing rate sequence 125.

Referring next to FIG. 1c, in an embodiment, the breathing pacer 20 is configured to time the breathing events to the person 90 with the breathing rate sequence 125 comprising breathing rates 125a, 125b such that a successive breathing rate 125b of the breathing rate sequence 125 is higher than a previous breathing rate 125a of the breathing rate sequence 125. This corresponds to a ramp-up of instructed breathing rates (ramp-up in time). There may be for example eight different breathing rates in a breathing rate sequence, 4 BRPM, 4.5 BRPM, 5 BRPM, 5.5 BRPM, 6 BRPM, 6.5 BRPM, 7 BRPM and 7.5 BRPM in the breathing rate sequence 125.

Referring next to FIG. 1d, in an embodiment, the breathing pacer 20 is configured to time the breathing events to the person 90 with the breathing rate sequence 125 comprising breathing rates 125a, 125b such that the breathing rate sequence 125 is a predetermined breathing rate sequence 125d. A predetermined breathing rate sequence 125d may be set to the breathing pacer 20, to the system 1 or to the data processing system 30, for example set to the memory of a data processing system 30, breathing pacer 20 or system 1. A predetermined breathing rate sequence 125d may also be based on a rule that determines the next breathing rate in the sequence based on previous results of the fitting, that is, the parameters 210 of the periodic biofeedback function 200, and the cumulative difference E 220 at each of the previous timepoints t1, t2.

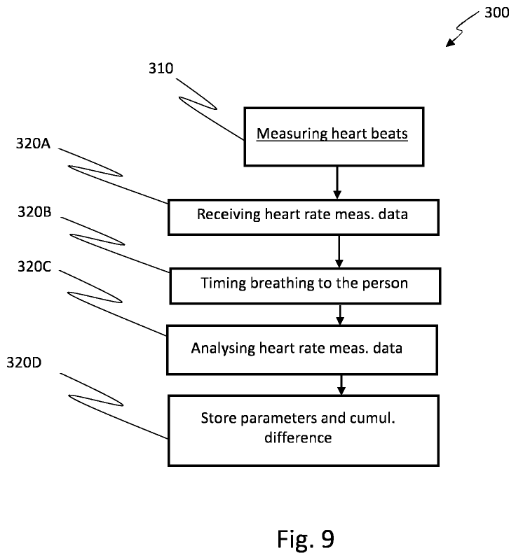

Next referring to FIG. 9 and also to FIG. 1a, as an aspect of the invention, a method 300 for monitoring health of a person 90 during a health monitoring session s is disclosed. The method 300 comprises: measuring (as step 310) heart beats of the person 90 for providing heart rate measurement data 110 with a heart rate sensor 10, receiving (as step 320A) the heart rate measurement data 110 from the heart rate sensor 10 into a data processing system 30. The method also comprises timing (as step 320B) breathing events to the person 90 with a breathing pacer 20 and with a breathing rate sequence 125 comprising breathing rates 125a, 125b. The health monitoring session s comprises a time window sequence wn of time windows w1, w2, such that each of the breathing rates of the breathing rate sequence 125 is associated with associated time window w1, w2 of the time window sequence wn. In other words, the health monitoring session s comprises a time window sequence wn, the time window sequence wn comprising time windows w1, w2, such that each of the breathing rates of the breathing rate sequence 125 is associated with associated time window w1, w2 of the time window sequence wn.

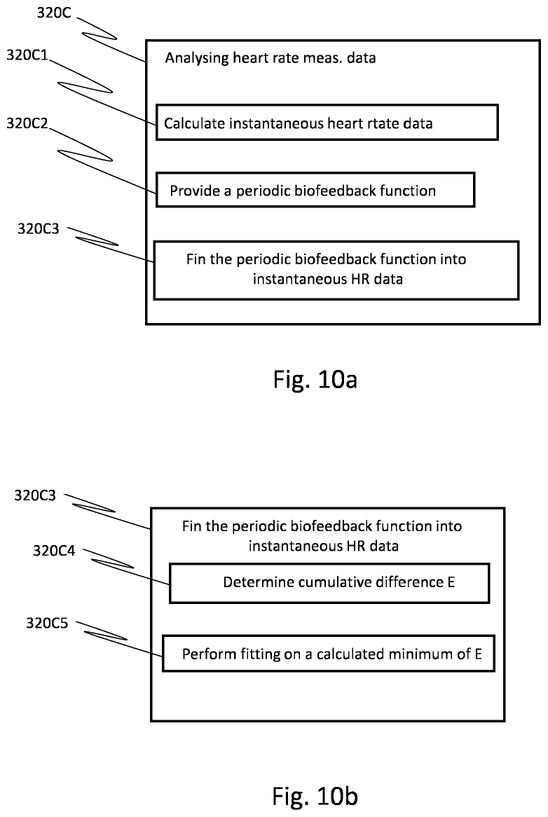

Referring next in more detail to FIG. 10a, the method also comprises analysing (as step 320C) the heart rate measurement data 110 in the data processing system 30 by
calculating (in step 320C1) instantaneous heart rate data 112 comprising heart rate of each measured heartbeat, calculating performed over each of the time windows w1, w2 in the health monitoring session s, each of the time windows w1, w2 comprising a timepoint t1, t2. The instantaneous heart rate data 112 is calculated based on the heart rate measurement data 110 received from the heart rate sensor 10,
providing (in step 320C2) a periodic biofeedback function 200 comprising parameters 210, the parameters 210 comprising an amplitude A 201, the periodic biofeedback function 200 comprising a cumulative difference E 220 relative to the instantaneous heart rate data 112,
fitting (in step 320C3) the periodic biofeedback function 200 into the instantaneous heart rate data 112 within each of the time windows w1, w2 to determine the parameters 210 of the periodic biofeedback function 200 at each of the timepoints t1, t2, and the cumulative difference E 220 at each of the timepoints t1, t2, and
storing (as step 320D) the parameters (210) of the periodic biofeedback function (200) at each of the timepoints t1, t2, and the cumulative difference E (220) at each of the timepoints t1, t2.

Storing (step 320D) may also comprise storing the breathing rates 125a, 125b associated with the time windows w1, w2 comprising the timepoints t1, t2.

The method 300 may also comprise the data processing system 30 providing the time window sequence wn such that the time window sequence wn comprises time windows w1, w2.

The method 300 may also comprise the data processing system 30 providing the breathing rate sequence 125 comprising the breathing rates 125a,125b such that each of the breathing rates 125a, 125b of the breathing rate sequence 125 is associated with associated time window w1, w2 of the time window sequence wn.

Various concepts of the method aspect of the invention are already defined in the system aspect of the invention related to system 1 above.

Referring next to FIG. 10b, as an embodiment, in the fitting, in step 320C3, the method 300 comprises determining, in step 320C4, the cumulative difference E 220 between the instantaneous heart rate data 112 and the periodic biofeedback function 200 within each of the time windows w1, w2 comprising the timepoints t1, t2, and performing, in step 320C5, the fitting based on a calculated minimum of the cumulative difference E 220 within each of the time windows w1, w2 to determine the parameters 210 of the periodic biofeedback function 200 at each of the timepoints t1, t2, the time windows w1, w2 comprising the timepoints t1, t2.

The method 300 may also comprise, after step 320C5, determining the cumulative difference E 220 at each the timepoints t1, t2 from the calculated minimum of the cumulative difference E 220 within each of the time windows w1, w2.

The method 300 may also comprise, after step 320C5, setting the value of the cumulative difference E 220 at each of the timepoints t1, t2 to the value of the calculated minimum of the cumulative difference E 220 within each of the time windows w1, w2. In other words, after the fitting, the cumulative difference E 220 may have a minimum value within each of the time windows w1,w2.

In an embodiment, in step 320C5, the method 300 comprises performing the fitting (as step 320C5) with a least squares method, a modified least squares method, a random search, an exhaustive search, or any combination thereof. Operation of these methods is discussed above in relation to system 1.

In an embodiment, and referring back to FIG. 4, in the method 300, the periodic biofeedback function f(t) 200 at each of the timepoints (t1, t2) comprises a sinusoidal function sin( ) such that f(t) is defined as f(t)=A sin(Pt−T)+M, in which A is the amplitude A 201 of the instantaneous heart rate data 112 within each of the time windows w1, w2, P is the angular frequency P 202 of the instantaneous heart rate data 112 within each of the time windows w1, w2, T is a time displacement, and M is a mean value M 205 of the instantaneous heart rate data 112 within each of the time windows w1, w2.

In an embodiment, and referring back to FIG. 5, in the method 300, the periodic biofeedback function f(t) 200 at each of the timepoints t1, t2 comprises a skewed sinusoidal function sk sin( ) such that f(t) is defined as f(t)=A sk sin(Pt−T)+M=A sin[(Pt−T)+k*sin(Pt−T)], in which A is the amplitude A 201 of the instantaneous heart rate data 112 within each of the time windows w1, w2, P is the angular frequency P 202 of the instantaneous heart rate data 112 within each of the time windows w1, w2, T is a time displacement, k is a skew factor, and M is a mean value M 205 of the instantaneous heart rate data 112 within each of the time windows w1, w2. The skew factor k can be set to configure the periodic biofeedback function waveform asymmetric so that, for example, rise time (from zero value to the positive or negative amplitude value or peak value) is shorter in time than the fall time from the amplitude value back to the zero value. This is advantageous as the inhalation is often somewhat shorter than the exhalation and thus the asymmetric waveform may easier be fit to the instantaneous heart rate data 112.

In an embodiment, as illustrated in FIG. 6b, the method 300 comprises providing feedback with the breathing pacer 20 within at least one of the time windows w1, w2, the feedback based on the parameters 210 of the periodic biofeedback function 200; or the cumulative difference E 220; or any combination thereof.

In an embodiment, as illustrated in FIG. 6b, the method 300 comprises determining a resonance score 230 from the amplitude A 201 of the periodic biofeedback function 200 and from the cumulative difference E 220 within at least one of the time windows w1, w2, and providing feedback to the person 90 within at least one of the time windows w1, w2 based on the resonance score 230 with the breathing pacer 20.

In an embodiment, as illustrated in FIG. 6b, the method 300 comprises determining a resonance score 230 from the amplitude A 201 of the periodic biofeedback function 200 and from the cumulative difference E 220 within each of the time windows w1, w2, and providing feedback to the person 90 by indicating to the person 90, with the breathing pacer 20, the breathing rate 125a, 125b having the best resonance score 230. Referring to FIG. 3b, the breathing rate 125a, 125b having the best resonance score 230 is denoted as 125MAX, and the highest resonance score 230 is denoted as 230MAX.

In an embodiment, the method 300 comprises determining the resonance score 230 based on the division of the amplitude A 201 of the periodic biofeedback function 200 by the average cumulative difference E_{AVE} 220 within the time window w. The average cumulative difference E_{AVE} 220 may be calculated by dividing the cumulative difference E by the number of discrete datapoints N used to determine the cumulative difference E as illustrated in relation to FIG. 3a.

In an embodiment, the method 300 comprises determining the resonance score 230 based on a tabulated and predetermined set of values for resonance score 230, the tabulated and predetermined set of values for resonance score 230 arranged based on the amplitude A 201 of the periodic biofeedback function 200 and cumulative difference E 220 within at least one of the time windows w1, w2.

In an embodiment, as illustrated in FIG. 6b, the method 300 comprises providing feedback visually 93; or audially 94; or haptically 95; or any combination thereof. Providing visual feedback may comprise showing a symbol or a text on a display device of the breathing pacer 20. For example, text “high vagus nerve stimulation” may indicate a high resonance score 230. Providing audial feedback may comprise emitting a sound from a loudspeaker of the breathing pacer 20. The tone of the sound (for example, frequency of the sound) may be inversely related to the resonance score 230 (high sound indicates poor vagus nerve stimulation and low sound a strong vagus nerve stimulation). Providing haptic feedback may comprise emitting a vibration from a linear resonant device of the breathing pacer 20 (for example, high frequency vibration indicates poor vagus nerve stimulation and low frequency vibration indicates a strong vagus nerve stimulation).

In an embodiment, as illustrated in FIG. 1b, in timing (as step 320B) the breathing events to the person 90 with the breathing pacer 20 and with the breathing rate sequence 125 comprising breathing rates 125a, 125b, a successive breathing rate 125b of the breathing rate sequence 125 is lower than a previous breathing rate 125a of the breathing rate sequence 125.

In an embodiment, as illustrated in FIG. 1c, in timing (as step 320B) the breathing events to the person 90 with the breathing pacer 20 and with the breathing rate sequence 125 comprising breathing rates 125a, 125b, a successive breathing rate 125b of the breathing rate sequence 125 is higher than a previous breathing rate 125a of the breathing rate sequence 125.

In an embodiment, as illustrated in FIG. 1d, in timing (as step 320B) the breathing events to the person 90 with the breathing pacer 20 and with the breathing rate sequence 125 comprising breathing rates 125a, 125b, the breathing rate sequence 125 is a predetermined breathing rate sequence 125d. A predetermined breathing rate sequence 125d may be set to the breathing pacer 20, to the system 1 or to the data processing system 30, for example set to the memory of a data processing system 30, breathing pacer 20 or system 1. A predetermined breathing rate sequence 125d may also be based on a rule that determines the next breathing rate in the sequence based on previous results of the fitting, that is, the parameters 210 of the periodic biofeedback function 200, and the cumulative difference E 220 at each of the previous timepoints t1, t2.

As an aspect of the invention, the method 300 defined above may be executed in the system 1 as defined above.

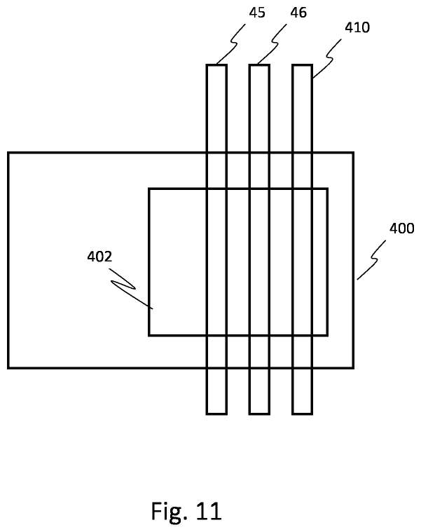

Next turning to FIG. 11, as an aspect of the present invention, a computer program 400 comprises executable instructions 402 which are configured to execute the steps of the method 300 in a computer 410, or in a mobile computing device 45 or network data server 46, or any combination thereof.

For the purposes of this text, the data processing system 30 may comprise digital electronics, analogue electronics, memory or memories, power unit like a battery, ASICs, FPGAs, digital busses, microcontrollers and microprocessors to arrange its various operations. The data processing system 30 may also comprise one or more input devices like a keyboard, a microphone or a touch screen display, and output devices like a LED, a display, a loudspeaker or a haptic device like a linear resonant actuator. The data processing system 30 may be arranged in a mobile computing device like a smartphone or tablet computer, for example through software and hardware means. Various communication standards and protocols like I2C, SPI, Ethernet, WLAN and Bluetooth may be employed for communications. If the heart rate measurement data from the heart rate sensor 10 is in analogue format, the data processing system 30 may also comprise analogue-to-digital converters to arrange conversion of an analogue signal into a digital signal for further processing.

The invention has been described above with reference to the examples shown in the figures. However, the invention is in no way restricted to the above examples but may vary within the scope of the claims.

1.-37. (canceled)

38. A system for monitoring health of a person during a health monitoring session (s), wherein the system comprises:
a heart rate sensor arranged to measure heart beats of the person for providing heart rate measurement data,
a breathing pacer configured to time breathing events to the person with a breathing rate sequence comprising different breathing rates,
the health monitoring session (s) comprising a time window sequence (wn), the time window sequence (wn) comprising time windows (w1, w2), such that each of the different breathing rates of the breathing rate sequence timed by the breathing pacer is associated with associated time window (w1, w2) of the time window sequence (wn),
the system comprises:
a data processing system configured, during a health monitoring session (s), to
receive (A) the heart rate measurement data from the heart rate sensor,
arrange (B) the breathing pacer to time the breathing events to the person in each of the different breathing rates,
analyse (C) the heart rate measurement data such that in the analysis, the data processing system is configured to:
calculate (C1) instantaneous heart rate data comprising heart rate of each measured heartbeat, calculating performed over each of the time windows (w1, w2) in the health monitoring session (s), each of the time windows (w1, w2) comprising a timepoint (t1, t2), the instantaneous heart rate data being calculated based on the heart rate measurement data received from the heart rate sensor,
provide (C2) a periodic biofeedback function comprising parameters and a cumulative difference E relative to the instantaneous heart rate data, the parameters of the periodic biofeedback function comprising an amplitude (A),
fit (C3) the periodic biofeedback function into the instantaneous heart rate data within each of the time windows (w1, w2) to determine
the parameters of the periodic biofeedback function at each of the timepoints (t1, t2), and
the cumulative difference (E) at each of the timepoints (t1, t2); and
determine (E) a resonance score based on the amplitude (A) of the periodic biofeedback function and from the cumulative difference E within each of the time windows (w1, w2).

39. The system according to claim 38, wherein the data processing system is further configured, during a health monitoring session (s), to
store (D) the parameters of the periodic biofeedback function, and the cumulative difference E at each of the timepoints (t1, t2).

40. The system according to claim 38, wherein the data processing system is further configured, during a health monitoring session (s), to
F) compare (F) the resonance scores of the time windows (w1, w2) to each other and determine the breathing rate having the best resonance score.

41. The system according to claim 38, wherein:
successive time windows (w1, w2) in the time window sequence (wn) have different breathing rates timed by the breathing pacer; or
successive time windows (w1, w2) in the time window sequence (wn) have decreasing breathing rates timed by the breathing pacer; or
successive time windows (w1, w2) in the time window sequence (wn) have increasing breathing rates timed by the breathing pacer.

42. The system according to claim 41, wherein the data processing system is further configured, during a health monitoring session (s), to
arrange (B1) the breathing pacer to operate at a first breathing rate in a first time window (w1);
analyse (C1) the heart rate measurement data within the first time window (w1);
determine (E1) the resonance score based on the amplitude A of the periodic biofeedback function and from the cumulative difference E within the first window (w1);
decrease (C2) the breathing rate of the breathing pacer and arrange the breathing pacer to operate at a decreased breathing rate in a subsequent time window (w2);
analyse (C2) the heart rate measurement data within the subsequent time window (w2);
determine (E2) the resonance score based on the amplitude A of the periodic biofeedback function and from the cumulative difference E within the subsequent window (w2); and
compare (F) the resonance scores of the first time window (w1) and the subsequent time window (w2) to each other and determine the breathing rate having the best resonance score; or
arrange (B1) the breathing pacer to operate at a first breathing rate in a first time window (w1);
analyse (C1) the heart rate measurement data within the first time window (w1);
determine (E1) the resonance score based on the amplitude A of the periodic biofeedback function and from the cumulative difference E within the first window (w1);
decrease (B2) the breathing rate of the breathing pacer and arrange the breathing pacer to operate at a decreased breathing rate in a subsequent time window (w2);
analyse (C2) the heart rate measurement data within the subsequent time window (w2);
determine (E2) the resonance score based on the amplitude A of the periodic biofeedback function and from the cumulative difference E within the subsequent window (w2);
repeat B2, C2 and E2 one or more time for two or more subsequent time windows (w1, w2) in the time window sequence (wn); and
compare F) the resonance scores of the first time window (w1) and the subsequent time windows (w2) to each other and determine the breathing rate having the best resonance score.

43. The system according to claim 41, wherein the data processing system is further configured, during a health monitoring session (s), to
arrange (B1) the breathing pacer to operate at a first breathing rate in a first time window (w1);
analyse (C1) the heart rate measurement data within the first time window (w1);
determine (E1) the resonance score based on the amplitude A of the periodic biofeedback function and from the cumulative difference E within the first window (w1);
increase (C2) the breathing rate of the breathing pacer and arrange the breathing pacer to operate at a increased breathing rate in a subsequent time window (w2);
analyse (C2) the heart rate measurement data within the subsequent time window (w2);
determine (E2) the resonance score based on the amplitude A of the periodic biofeedback function and from the cumulative difference E within the subsequent window (w2); and
compare (F) the resonance scores of the first time window (w1) and the subsequent time window (w2) to each other and determine the breathing rate having the best resonance score; or
arrange (B1) the breathing pacer to operate at a first breathing rate in a first time window (w1);
analyse (C1) the heart rate measurement data within the first time window (w1);
determine (E1) the resonance score based on the amplitude A of the periodic biofeedback function and from the cumulative difference E within the first window (w1);
increase (B2) the breathing rate of the breathing pacer and arrange the breathing pacer to operate at a increased breathing rate in a subsequent time window (w2);
analyse (C2) the heart rate measurement data within the subsequent time window (w2);
determine (E2) the resonance score based on the amplitude A of the periodic biofeedback function and from the cumulative difference E within the subsequent window (w2);
repeat B2, C2 and E2 one or more time for two or more subsequent time windows (w1, w2) in the time window sequence (wn); and
compare (F) the resonance scores of the first time window (w1) and the subsequent time windows (w2) to each other and determine the breathing rate having the best resonance score.

44. The system according to claim 38, wherein the fitting (C3), the data processing system is configured to:
determine the cumulative difference E between the instantaneous heart rate data and the periodic biofeedback function within each of the time windows (w1, w2), the time windows (w1, w2) comprising the timepoints (t1, t2), and
perform the fitting (C3) based on a calculated minimum of the cumulative difference E within each of the time windows (w1, w2) to determine the parameters of the periodic biofeedback function at each of the timepoints (t1, t2), the time windows (w1, w2) comprising the timepoints (t1, t2).

45. The system according to claim 38, wherein fitting (C3), in determining the parameter indicating the amplitude A of the periodic biofeedback function at each of the timepoints (t1, t2), the data processing system is arranged to
determine a minimum instantaneous heart rate and a maximum instantaneous heart rate of instantaneous heart rate data within each of the time windows (w1, w2),
subtract the minimum instantaneous heart rate from the maximum instantaneous heart rate,
divide the value of subtraction by 2, and
determine the parameter indicating the amplitude A of the periodic biofeedback function at each of the timepoints (t1, t2) based on the value of the division.

46. The system according to claim 38, wherein the parameters of the periodic biofeedback function comprise an angular frequency (P), and in fitting (C3), in determining the parameter indicating the angular frequency (P) of the periodic biofeedback function at each of the timepoints (t1, t2), the data processing system is arranged to:
determine a cycle time (TD) of the instantaneous heart rate data within each of the time windows (w1, w2), and
determine the parameter indicating the angular frequency (P) of the periodic biofeedback function at each of the timepoints (t1, t2) based on the cycle time (TD).

47. The system according to claim 38, wherein:
the parameters of the periodic biofeedback function comprise a mean M of the periodic biofeedback function, and
in fitting (C3), in determining the mean (M) of the periodic biofeedback function at each of the timepoints (t1, t2), the data processing system is arranged to:
determine a mean value of the instantaneous heart rate data within each of the time windows (w1, w2), and
determine the mean (M) of the periodic biofeedback function at each of the timepoints (t1, t2) based on the mean value.

48. The system according to claim 38, wherein:
the periodic biofeedback function f(t) at each of the timepoints (t1, t2) comprises a sinusoidal function sin( ) such that f(t) is defined as f(t)=A sin(Pt−T)+M, in which
A is the amplitude A of the instantaneous heart rate data within each of the time windows (w1, w2),
P is the angular frequency P of the instantaneous heart rate data within each of the time windows (w1, w2),
T is a time displacement, and
M is a mean value M of the instantaneous heart rate data within each of the time windows (w1, w2); or
the periodic biofeedback function f(t) at each of the timepoints (t1, t2) comprises a skewed sinusoidal function sk sin( ) such that f(t) is defined as f(t)=A sk sin(Pt−T)+M=A sin[(Pt−T)+k*sin(Pt−T)], in which
A is the amplitude A of the instantaneous heart rate data within each of the time windows (w1, w2),
P is the angular frequency P of the instantaneous heart rate data within each of the time windows (w1, w2),
T is a time displacement,
k is a skew factor, * is a multiplication operator, and
M is a mean value M of the instantaneous heart rate data within each of the time windows (w1, w2).

49. The system according to claim 38, wherein
the breathing pacer is configured to provide feedback to the person within at least one of the time windows (w1, w2) based on the resonance score; or
the breathing pacer is configured to provide feedback to the person by indicating to the person the breathing rate having the best resonance score.

50. The system according to claim 41, wherein
the breathing pacer comprises third software means executable on a mobile computing device, and
the third software means are functionally connectable with the data processing system, and:
the third software means comprises computer-executable instructions for providing feedback based on:
the parameters of the periodic biofeedback function; or
the cumulative difference E; or
any combination thereof.

51. The system according to claim 38, wherein the breathing pacer is arranged to time the breathing events to the person by indicating to the person:
a start of an inhalation of each breathing cycle; or
an end of an inhalation of each breathing cycle; or
a start of an exhalation of each breathing cycle; or
an end of an exhalation of each breathing cycle; or
any combination thereof.

52. The system according to claim 38, wherein:
the data processing system comprises first software means executable on a mobile computing device, the first software means being functionally connectable with the heart rate sensor, and
the first software means comprises computer-executable instructions to (A) receive the heart rate measurement data, (B) arrange the breathing pacer to time the breathing events, (C) analyse the heart rate measurement data, and (D) store the parameters and the cumulative difference E.

53. The system according to claim 41, wherein:
the data processing system comprises second software means executable on a network data server,
the first software means and the second software means are configured to exchange data over a network connection, and
the second software means comprise computer-executable instructions for performing at least one of the steps of (A) receive the heart rate measurement data, (B) arrange the breathing pacer to time the breathing events, (C) analyse the heart rate measurement data and (D) store the parameters and the cumulative difference (E).

54. The system according to claim 38, wherein the breathing pacer is configured to time the breathing events to the person with the breathing rate sequence comprising breathing rates such that:
a successive breathing rate of the breathing rate sequence is lower than a previous breathing rate of the breathing rate sequence; or
a successive breathing rate of the breathing rate sequence is higher than a previous breathing rate of the breathing rate sequence; or
the breathing rate sequence is a predetermined breathing rate sequence.

55. A method for monitoring health of a person during a health monitoring session (s), wherein the method comprises:
measuring heart beats of the person for providing heart rate measurement data with a heart rate sensor,
receiving the heart rate measurement data from the heart rate sensor into a data processing system,
timing breathing events to the person with a breathing pacer and with a breathing rate sequence comprising different breathing rates, the health monitoring session (s) comprising a time window sequence (wn) of time windows (w1, w2), such that each of the different breathing rates of the breathing rate sequence timed by the breathing pacer is associated with associated time window (w1, w2) of the time window sequence (wn),
analysing the heart rate measurement data in the data processing system by
calculating instantaneous heart rate data comprising heart rate of each measured heartbeat, calculating performed over each of the time windows (w1, w2) in the health monitoring session (s), each of the time windows (w1, w2) comprising a timepoint (t1, t2), the instantaneous heart rate data being calculated based on the heart rate measurement data received from the heart rate sensor,
providing a periodic biofeedback function comprising parameters, the parameters comprising an amplitude A, the periodic biofeedback function comprising a cumulative difference E relative to the instantaneous heart rate data,
fitting the periodic biofeedback function into the instantaneous heart rate data within each of the time windows (w1, w2) to determine the parameters of the periodic biofeedback function at each of the timepoints (t1, t2), and the cumulative difference (E) at each of the timepoints (t1, t2), and
determining a resonance score from the amplitude A of the periodic biofeedback function and from the cumulative difference E within each of the time windows (w1, w2).

56. A processor-readable medium storing instructions which, when executed by at least one processor of an apparatus, cause the apparatus at least to perform the method according to claim 55.
